(19) United States
(12) Patent Application Publication
(10) Pub. No.: US 2024/0184952 A1
(43) Pub. Date: Jun. 6, 2024

(54) A METHOD AND A SYSTEM FOR PREDICTING THE PROPERTIES OF COATING LAYERS AND SUBSTRATES COMPRISING SAID COATING LAYERS

(71) Applicant: BASF Coatings GmbH, Muenster (DE)

(72) Inventors: Marc THOMAS, Muenster (DE); Klaus-Juergen KANNGIESSER, Muenster (DE); Markus MUNDUS, Muenster (DE); Michaela LIESE, Muenster (DE)

(21) Appl. No.: 18/549,817

(22) PCT Filed: Mar. 1, 2022
(86) PCT No.: PCT/EP22/55125

§ 371 Date: Sep. 8, 2023

(30) Foreign Application Priority Data
Mar. 17, 2021 (EP) 21163159.3

(51) Int. Cl. G06F 30/20

(52) U.S. Cl. CPC G06F 30/20

(57) ABSTRACT

Disclosed herein are a computer-implemented method, a system, and a computer program product for predicting the properties of a coating layer CL or transmission and/or reflection properties of a substrate coated with a coating layer CL and optionally at least one further coating layer. Further disclosed herein is a method of using the computer-implemented method for screening coating layers CL or coated substrates including at least one coating layer CL and optionally at least one further coating layer. Additionally disclosed herein are a substrate coated with a coating layer CL, at least one further coating layer, and a client device for generating a request to initiate the prediction of at least one property of a coating layer CL or at least one transmission and/or reflection property of the substrate.

### FIELD

Aspects described herein generally relate to methods and systems for predicting the properties of a coating layer or the transmission and/or reflection properties of coated substrates. More specifically, aspects described herein relate to the prediction of properties of a coating layer CL or the transmission and/or reflection properties of substrates being coated with a coating layer CL and optionally at least one further coating layer by determining a measure indicating the permittivity of the coating layer CL. The determined measure may then be used, optionally in combination with a measuring indicating the permittivity of further coating layers being present on the substrate, for predicting the transmission and/or reflection properties of such coated substrates.

### BACKGROUND

For improving automotive safety, radar devices that measure distances and warn the driver if the automobile approaches an object have become a new standard. Such radar devices may be provided at various parts of the automobile, for example, behind the radiator grill, the bumper, and the like. The development of cars driving autonomously will further increase the need of having various radar sensitive sensors. For future cars about 80 sensors may be installed in a car which measure distances and velocities of surrounding objects in all directions.

It is often desirable to conceal the sensors, such as radar sensors, invisibly behind a trim part, for example a bumper of the motor vehicle, in order to reduce the negative impact of such sensors on the overall visual appearance of the car. Such trim parts are normally coated with essentially the same pigmented coating compositions (i.e. basecoat compositions) used to paint the car body in order to create a uniform high-class optical appearance to the observer. Today it is common standard that most automotive basecoat compositions comprise effect pigments such as metallic or pearlescent pigments. These platelet-like pigments orient in a parallel manner with respect to the coating substrate. In case of metallic platelets, they act as little mirrors leading to high metallic gloss and flop effects (change of the lightness when viewed under different angles of incidence of observance) combined with an outstanding hiding power. In most cases the metallic pigments are aluminum pigments giving rise to silver-colored metallic coatings. In case of pearlescent pigments interference colors are produced and the coatings exhibit optical depth.

Due to the concealment of the radar sensors behind trim parts, the emitted radar waves as well as the radar waves reflected from surrounding objects must radiate through the trim part. However, part of the emitted radar waves is reflected on the trim part. This reflection leads, on the one hand, to a reduction of the range of the radar sensor and, on the other hand, to a reduced performance of angle-resolving radar sensors due to the interference signal formed from the reflected radar waves. Additionally, coating layers applied on the trim part substrate may absorb the emitted radar waves, thus further reducing the range of the radar sensor. In this respect, it is well known that a coating containing metallic effect pigments such as aluminum pigments may result in a high reflection and absorption of emitted radar waves, thus resulting in non-acceptable damping of the emitted radar signal.

Radar waves used for sensors typically lie in the frequency range of 65 to 85 GHz, which corresponds to a wavelength range of about 4 to 5 mm. Although these wavelengths are much larger than the size of the effect pigments or the thickness of the coating, the observed damping is due to the very high electrical conductivity of the interior of such aluminum pigments leading to the induction of a counter electromagnetic wave which is resulting in the observed damping of the radar wave. In order to ensure sufficient performance of the radar sensor mounted behind a coated substrate, the dampening effect observed by the coated substrate must be below 3 dB, preferably below 2 dB of the emitted radar intensity when measured under perpendicular angle of incidence.

To overcome these adverse effects, it is known to use precisely defined geometries as well as highly accurately defined dielectric properties of the trim parts in combination with paint layers containing special pigments which do not significantly dampen the emitted radar intensity.

The use of special paint layers, precisely defined application processes and precisely defined geometries of trim parts, however, results in significantly higher costs for manufacturing such coated substrates. Moreover, such coated substrates cannot be repaired by simply overcoating the damaged part as done routinely during refinish processes because such overcoating would significantly change the optimized system of trim part and paint layer with respect to the damping of the emitted radar intensity.

It would therefore be desirable to use efficient computer-based methods and systems which are able to calculate the dampening effect of coating layers, in particular basecoat layers, and coated objects comprising at least one coating layer, in particular at least one basecoat layer, thus allowing to screen existing coating formulations, in particular basecoat formulations, with regard to their suitability for the coating of trim parts mounted in front of devices emitting electromagnetic radiation and detecting the reflected electromagnetic radiation, such as radar sensors, in order to avoid the use of expensive paint layers comprising special effect pigments which have to be applied under very specific conditions in order to reduce the dampening of the emitted electromagnetic radiation, such as the radar intensity. Such methods and systems would also render it possible to repair such trim parts by commonly used refinish processes.

### DEFINITIONS

“Data driven model” may refer to a model at least partially derived from data. Use of a data driven model can allow describing relations, that cannot be modelled by physico-chemical laws. The use of data driven models can allow to describe relations without solving equations from physico-chemical laws. This can reduce computational power and can improve speed. The data driven model may be derived from statistics (Statistics 4th edition, David Freedman et al., W. W. Norton & Company Inc., 2004). The data driven model may be derived from Machine Learning (Machine Learning and Deep Learning frameworks and libraries for large-scale data mining: a survey, Artificial Intelligence Review 52, 77-124 (2019), Springer). The data driven model may comprise empirical or so-called “black box models”. Empirical or “black box” model may refer to models being built by using one or more of machine learning, deep learning, neural networks, or other form of artificial intelligence. The empirical or “black box” model may be any model that yields a good fit between training and test data. Alternatively, the data driven model may comprise a rigorous or “white box” model. A rigorous or “white box” model refers to models based on physico-chemical laws. The physico-chemical laws may be derived from first principles. The physico-chemical laws may comprise one or more of chemical kinetics, conservation laws of mass, momentum and energy, particle population in arbitrary dimension, physical and/or chemical relationships. The rigorous or “white box” model may be selected according to the physico-chemical laws that govern the respective problem. The data driven model may also comprise hybrid models. “Hybrid model” refers to a model that comprises white box models and black box models, see e.g. review paper of Von Stoch et al., 2014, Computers & Chemical Engineering, 60, Pages 86 to 101.

“Digital representation” may refer to a representation of the coating layer CL, the coated substrate, all further coating layer(s) CL-x being present in addition to the coating layer CL and historical coating layers in a computer readable form. In particular, the digital representation of the coating layer CL and historical coating layers may, e.g. be a composition of the coating material used to prepare the respective coating layer, data on at least one property of the coating material used to prepare the respective coating layer, data on at least one property of the respective coating layer or any combination thereof. The digital representation of the coated substrate may, e.g. be the layer thickness of the substrate, the layer thickness of the coating layer CL and further coating layer(s) CL-x being present, a measure indicating the permittivity of the substrate or any combination thereof. The digital representation of further coating layer(s) CL-x may, e.g. be a composition of the coating material used to prepare each further coating layer CL-x, data on at least one property of each coating material used to prepare the further coating layer(s) CL-x, data on at least one property of further coating layer(s) CL-x, a measure indicating the permittivity of the further coating layer(s) CL-x or any combination thereof.

“Machine Learning” may refer to computer algorithms that improve through experience, machine Learning algorithms build on a model based on sample data, often described as training data.

“Communication interface” may refer to a software and/or hardware interface for establishing communication such as transfer or exchange of signals or data. Software interfaces may be e. g. function calls, APIs. Communication interfaces may comprise transceivers and/or receivers. The communication may either be wired, or it may be wireless. Communication interface may be based on or it supports one or more communication protocols. The communication protocol may a wireless protocol, for example: short distance communication protocol such as Bluetooth®, or WIFI, or long distance communication protocol such as cellular or mobile network, for example, second-generation cellular network (“2G”), 3G, 4G, Long-Term Evolution (“LTE”), or 5G. Alternatively, or in addition, the communication interface may even be based on a proprietary short distance or long distance protocol. The communication interface may support any one or more standards and/or proprietary protocols.

“Computer processor” refers to an arbitrary logic circuitry configured to perform basic operations of a computer or system, and/or, generally, to a device which is configured for performing calculations or logic operations. In particular, the processing means, or computer processor may be configured for processing basic instructions that drive the computer or system. As an example, the processing means or computer processor may comprise at least one arithmetic logic unit (“ALU”), at least one floating-point unit (“FPU)”, such as a math coprocessor or a numeric coprocessor, a plurality of registers, specifically registers configured for supplying operands to the ALU and storing results of operations, and a memory, such as an L1 and L2 cache memory. In particular, the processing means, or computer processor may be a multicore processor. Specifically, the processing means, or computer processor may be or may comprise a Central Processing Unit (“CPU”). The processing means or computer processor may be a (“CISC”) Complex Instruction Set Computing microprocessor, Reduced Instruction Set Computing (“RISC”) microprocessor, Very Long Instruction Word (“VLIW”) microprocessor, or a processor implementing other instruction sets or processors implementing a combination of instruction sets. The processing means may also be one or more special-purpose processing devices such as an Application-Specific Integrated Circuit (“ASIC”), a Field Programmable Gate Array (“FPGA”), a Complex Programmable Logic Device (“CPLD”), a Digital Signal Processor (“DSP”), a network processor, or the like. The methods, systems and devices described herein may be implemented as software in a DSP, in a micro-controller, or in any other side-processor or as hardware circuit within an ASIC, CPLD, or FPGA. It is to be understood that the term processing means or processor may also refer to one or more processing devices, such as a distributed system of processing devices located across multiple computer systems (e.g. cloud computing) and is not limited to a single device unless otherwise specified.

“Substrate being coated with a coating layer CL and optionally at least one further coating layer CL-x” refers to a substrate comprising a coating layer CL and optionally at least one further coating layer CL-x. The coating layer CL does not necessarily be in direct contact with the substrate, i.e. at least one further coating layer CL-x can be present between the substrate and the coating layer CL. Moreover, at least one further coating layer may be present on top of the coating layer CL, i.e., on the side of the coating layer CL facing away from the substrate. The coating layer CL and all further coating layers CL-x being present on the substrate in addition to the coating layer CL are preferably cured. “Curing” of a coating film is understood to mean the conversion of such a film to the ready-to-use state, i.e. to a state in which the substrate provided with the respective coating film can be transported, stored, and used as intended. More particularly, a cured coating film is no longer soft or tacky, but has been conditioned as a solid coating film which does not undergo any further significant change in its properties, such as hardness or adhesion on the substrate, even under further exposure to curing conditions as described below.

“Substrate being transparent to electromagnetic radiation having a frequency of 22 to 300 GHz” in the context of the invention refers to substrates which demonstrate a % in transmission of at least 70 to electromagnetic radiation in the frequency range of 22 to 300 GHz, preferably in the frequency range of 22 to 144 GHz. The % in transmission can be determined, for example, by mounting the substrate between electromagnetic radiation transmitter and receiver antennas, measuring the amount of transmitted signal that was not detected at the receiver (denoted IL in the following formula) and calculating the % in transmission according to the following formula:

% transmission=100×10^{IL/10}

“Vehicle identification data” refers to data which can be used to identify a vehicle based on said data. Such data may include the vehicle identification number (VIN), part of the VIN, a manufacturer of the vehicle, a manufacturer plant site of the vehicle, make, model or model year of the vehicle, paint color code, production sequence of the vehicle or a combination thereof

“Pigmented coating layer” in the context of the present invention denotes a cured coating layer comprising at least one pigment and/or dye. Pigments can be selected from coloring and/or effect pigments. “Basecoat layer” may refer to a cured color-imparting intermediate coating layer commonly used in automotive painting and general industrial painting. The basecoat material used to prepare the basecoat layer may be formulated as a solid color (straight shade) or effect color coating. “Effect color coatings” generally contain at least one effect pigment and optionally other colored pigments or spheres which give the desired color and effect. “Straight shade” or “solid color coatings” primarily contain colored pigments and exhibit no visible flop or two-tone metallic effect. The basecoat layer is formed by applying the basecoat material to a metal or plastic substrate optionally pre-treated with a filler layer, a primer-surfacer layer, or a primer layer, drying the formed basecoat film, and curing the dried film. A “filler layer” (primer-surfacer layer) is describing an intermediate layer used to fill out the irregularities of the substrate, to support corrosion resistance and adhesion as well as to provide protection from mechanical exposure such as stone chipping. A “primer layer” is describing the first layer of a multilayer coating which is applied onto the substrate and is used to provide improved adhesion for the multilayer coating. Moreover, the primer layer can provide improved corrosion protection, for example on metallic substrates. The term “drying of the basecoat film” refers to the vaporization of organic solvents and/or water present in a coating material after application and results in a coating film having a lower amount of solvent than the coating material. Said film it is no longer free-flowing, but is still soft and/or tacky, and in some cases only partly dried. The basecoat layer may be overcoated with a cured clearcoat layer, which protects the basecoat layer against weathering as well as mechanical and chemical attack. If the basecoat layer is overcoated with a clearcoat layer, the basecoat and clearcoat layer can also be jointly cured after application and optional drying of the clearcoat material.

Appearance” used herein refers to the perception in which the spectral and geometric aspects of a surface is integrated with its illuminating and viewing environment. In general, appearance includes visual texture such as coarseness caused by effect pigments, sparkle, or other visual effects of a surface, especially when viewed from varying viewing angles and/or with varying illumination angle

“Data derived from the chemical composition of the coating material” may refer to data being apparent from the chemical composition of the coating material. In particular, this may be, e.g. the type and amount of each ingredient being present in the coating material.

“Computer readable medium” may refer to physical and other computer-readable media for carrying or storing computer-executable instructions and/or data structures. Such computer-readable media can be any available media that can be accessed by a general-purpose or special-purpose computer system. Computer-readable media may include physical storage media that store computer-executable instructions and/or data structures. Physical storage media include computer hardware, such as RAM,

ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

“Database” may refer to a collection of related information that can be searched and retrieved. The database can be a searchable electronic numerical, alphanumerical, or textual document; a searchable PDF document; a Microsoft Excel® spreadsheet; or a database commonly known in the state of the art. The database can be a set of electronic documents, photographs, images, diagrams, data, or drawings, residing in a computer readable storage media that can be searched and retrieved. A database can be a single database or a set of related databases or a group of unrelated databases. “Related database” means that there is at least one common information element in the related databases that can be used to relate such databases.

“Adjustment tool” may refer to a part of a graphical user interface which allows to modify the digital representation D₁, D₂ and optionally D₃₋ₓ provided in step (i) of the inventive method. The adjustment tool may comprise at least one modulator for each digital representation D₁, D₂ and optionally D₃₋ₓ provided in step (i).

“Client device” may refer to a computer or a program that, as part of its operation, relies on sending a request to another program or a computer hardware or software that accesses a service made available by a server. The server may or may not be located on another computer.

### SUMMARY

To address the above-mentioned problems in a perspective the following is proposed: A computer-implemented method for predicting the properties of a coating layer CL or the transmission and/or reflection properties of a substrate being coated with a coating layer CL and optionally at least one further coating layer CL-x, said method comprising the steps of:
(i) providing to a computer processor via a communication interface a digital representation D₁ of the coating layer CL, optionally a digital representation D₂ of the coated substrate and optionally a digital representation D₃₋ₓ of each further coating layer CL-x being present on the substrate in addition to the coating layer CL;
(ii) providing to the computer processor via the communication interface a data driven model parametrized on
   digital representations Dₕ of historical coating layers, and
   historical measures indicating the permittivity of said coating layers;
(iii) determining with the computer processor a measure indicating the permittivity of the coating layer CL based on
   the data driven model provided in step (ii), and
   the digital representation D₁ of the coating layer CL;
(iv) optionally determining with the computer processor a measure indicating the permittivity of at least one further coating CL-x layer being present on the substrate in addition to the coating layer CL based on
   the data driven model provided in step (ii), and
   the digital representation D₃₋ₓ of the further coating layer CL-x;
(v) optionally determining with the computer processor at least one transmission and/or reflection property of the coated substrate based on
   the measure indicating the permittivity of the coating layer CL provided in step (iii),
   optionally the digital representation D₃₋ₓ of each further coating layer being present on the substrate in addition to the coating layer CL or the measure indicating the permittivity of the at least one further coating layer CL-x provided in step (iv) optionally in combination with the digital representation D₃₋ₓ of further coating layers for which the measure indicating the permittivity was not provided in step (iv), and
   the digital representation D₂ of the coated substrate;
(vi) providing via the communication interface the determined measure indicating the permittivity of the coating layer CL and/or the determined at least one transmission and/or reflection property of the coated substrate.

The proposed method greatly reduces the time to obtain properties of the coating layer CL or transmission and/or reflection properties of coated substrates, by reducing the necessity of measuring said properties for each coating layer or coated substrate. In addition, the proposed method can be used to screen existing coating formulations and multilayer coatings according to at least one predefined criterion, for example the permittivity or the dampening of transmission and/or reflection at frequencies commonly used in connection with radar sensing devices in the automotive industry, thus allowing to select suitable coating formulations without performing extensive experiments that are otherwise required to determine whether the criterion is fulfilled.

Further disclosed is:

A computing apparatus comprising:
a communication interface;
a processing module comprising at least one computer processor; and
a memory storing instructions that, when executed by the processing module, configure the system to perform the steps of the computer implemented method of the invention disclosed therein.

Further disclosed is:

A non-transitory computer-readable storage medium, the computer-readable storage medium including instructions that when executed by a computer, cause the computer to perform the steps of the computer implemented method of the invention disclosed therein.

The disclosure applies to the methods, computer apparatuses, computer programs, computer readable non-transitory media, computer program products disclosed herein alike. Therefore, no differentiation is made between methods, computer apparatuses, computer programs, computer readable non-volatile storage media or computer program products. All features which are disclosed in connection with the computer implemented method of the invention are equally applicable to the computer apparatuses, computer programs, computer readable non-transitory storage media, and computer program products disclosed herein.

Further disclosed is a system comprising at least one coating layer CL and at least one measure indicating the permittivity of said at least one coating layer CL, wherein said measure indicating the permittivity is determined according to the method disclosed therein.

Further disclosed is the use of the computer implemented method of the invention for screening coating layers CL or coated substrates comprising at least one coating layer CL and optionally at least one further coating layer CL-x according to at least one criterion. In one example, the at least one criterion is a predefined range or value of a measure indicating the permittivity, in particular a predefined permittivity range or permittivity value. In another example, the at least one criterion is a predefined transmission and/or reflection tolerance, as previously described. This allows to screen existing coating formulations and multilayer coatings on substrates, especially plastic substrates, with respect to their use in combination with radar sensing devices. Thus, the use of special pigments, special substrate shapes or defined multilayer coatings is no longer necessary to prepare coated substrates having a visually appealing impression and being suitable for use in connection with radar sensing devices.

Further disclosed is a substrate being coated with a coating layer CL and at least one further coating layer CL-x, wherein the transmission and/or reflection property of the substrate was derived according to the computer implemented method of the invention disclosed therein.

Further disclosed is a client device for generating a request to initiate the prediction of at least one property of a coating layer CL or at least one transmission and/or reflection property of a substrate being coated with a coating layer CL and optionally at least one further coating layer CL-x at a server device, wherein the client device is configured to provide a digital representation D₁ of the coating layer CL, optionally a digital representation D₂ of the coated substrate, optionally a digital representation D₃₋ₓ of each further coating layer CL-x being present on the substrate in addition to the coating layer CL and optionally a property tolerance to a server device.

### EMBODIMENTS

### Embodiments of the Inventive Method

The transmission and/or reflection property may be predicted at frequencies commonly used in connection with radar sensing devices in the automotive industry (called radar transmission and/or reflection property hereinafter). A preferred radar transmission and/or reflection property is the dampening of the radar transmission and/or reflection. The radar transmission and/or reflection property may be particularly interesting when the coated substrate is to be mounted before radar sensing devices commonly used in the automotive industry because such coated substrates must fulfill predefined transmission and/or reflection criteria in order to prevent a negative impact on the performance of the radar sensing device.

The transmission and/or reflection property may be a classifier, such as “suitable” or “non suitable”. This may be derived from a predefined threshold, in particular a predefined maximal dampening of the radar transmission and/or reflection.

In an aspect, the substrate may be transparent to electromagnetic radiation having a frequency of 22 to 300 GHz, preferably a frequency of 22 to 144 GHz. Suitable substrates may comprise or consist of polycarbonate, blends of polycarbonate and polybutylene terephthalate, elastomer-modified polypropylene, blends of polypropylene and ethylene-propylene-diene rubber, acrylonitrile butadiene styrene copolymer, blends of acrylonitrile butadiene styrene copolymer with polycarbonate, acryl ester styrene acrylonitrile copolymer, polyamide and blends thereof, polyurethanes, blends of polycarbonate and polyethylene terephthalate, polybutylene terephthalate and mixtures thereof. Use of such transparent substrates reduces the negative impact on the electromagnetic radiation that propagates through the substrate.

In an aspect, the coating layer CL may be selected from pigmented coating layers, preferably from basecoat layers. The use of pigmented coating layers, in particular basecoat layers, provides coated substrates having a visually appealing impression.

The substrate may be coated with exactly one coating layer, i.e. the coating layer CL, or the substrate may be coated with at least two coating layers, i.e. the coating layer CL and at least one further coating layer CL-x. In an aspect, the substrate may be coated with a multilayer coating comprising the following layers, in particular in the stated order: optionally at least one primer layer PL, the coating layer CL, in particular being a basecoat layer, optionally at least one further basecoat layer being different from the coating layer CL and at least one clearcoat layer CL. Such multilayer coatings are commonly used in the automotive industry to provide high quality visually appealing impressions of the coated substrates.

### Step (i):

In step (i), the digital representation D₁ and optionally D₂ and D₃₋ₓ are provided via a communication interface to at least one processor. The digital representation(s) in step (i) can be provided by manually inputting the respective data, by importing the respective data from a computer readable medium, such as a file, a database or a cloud, or by obtaining the respective data from a measuring device, such as a spectrophotometer, and providing the obtained data via the communication interface. The communication interface may comprise a display, preferably a display having a graphical user interface. The GUI may facilitate data input, for example by providing adjustment tools which can be used to enter the respective data or by providing buttons for data import.

In an aspect, the step of providing the digital representation D₁ of the coating layer CL and/or the digital representation D₂ of the coated substrate and/or the digital representation D₃₋ₓ of each further coating layer CL-x in step (i) includes providing vehicle identification data, obtaining the digital representation D₁ and/or D₂ and/or D₃₋ₓ based on the provided vehicle identification data, and providing said obtained digital representation D₁ and/or D₂ and/or D₃₋ₓ. The vehicle identification data may be inputted manually by a user, may be selected from a list of available vehicle identification data, or may be provided by scanning a respective tag, for example a bar code or a QR code. Obtaining the digital representation D₁ and/or D₂ and/or D₃₋ₓ may further be defined as searching a database for said digital representations based on the inputted vehicle identification data. Use of the vehicle identification data can increase the user comfort with respect to providing the required digital representations D₁, D₂ and optionally D₃₋ₓ in step (i), because said data does not have to be inputted manually.

In an aspect, the step of providing the digital representation D₁ of the coating layer CL may comprise:
providing data derived from the chemical composition of the coating material used to prepare the coating layer CL,
optionally providing data on least one physical property of the coating material used to prepare the coating layer CL, and
optionally providing data on least one physical property of the coating layer CL.

Providing the aforementioned data in step (i) may include manual input of said data, importing said data from a computer readable medium or manipulating adjustment tools displayed via the communication interface to a user. Data derived from the chemical composition of the coating material may include the type and amount of each pigment, in particular effect pigment, being present in the coating material. Such data may be provided by importing the formulation of said coating material from a computer readable medium, such as a database, a computer file, or the like. With particular preference, the formulation is imported from at least one database connected via a communication interface to the at least one processor. Data on the at least one physical property may refer to data being obtained during determination of said property. Such physical property data of the coating material may include, for example, the solids content. Such physical property data of the coating layer CL may include, for example, appearance data, such as flop index data, color values, data describing the orientation of effect pigments, preferably aluminum pigments, within the coating layer CL, data acquired during the application of the coating material used to prepare the coating layer CL and combinations thereof. With particular preference, data on the at least one physical property of the coating layer CL includes flop index data. It may be preferred if data on at least one physical property of the coating material and data on at least one physical property the coating layer CL is provided in combination with data derived from the chemical composition of the coating material, because this may increase the accuracy of the determination of the measure indicating the permittivity of the coating layer CL and therefore also the accuracy of the proposed method.

In an aspect, the step of providing the digital representation D₂ of the coated substrate may include providing the thickness of the substrate, a measure indicating the permittivity of the substrate, the layer thickness of the coating layer CL and optionally of each further coating layer CL-x being present on the substrate in addition to the coating layer CL. The term “layer thickness” refers to the dry film thickness of the coating layer CL and—if present—the further coating layer(s) CL-x. The thickness of the substrate as well as the layer thickness of the coating layer CL and optionally each further coating layer CL-x being present in addition to the coating layer CL may be inputted manually or may be imported from a computer readable medium, such as a file or a database. Such data may also be retrieved from a database via the vehicle identification number as previously described. The measure indicating the permittivity of the substrate may be determined by measurement or a standard measure indicating the permittivity of the substrate may be used. The term “standard measure indicating the permittivity of the substrate” refers to a measure indicating the permittivity, which is representative for substrates commonly used in the respective application, such as the automotive industry.

In an aspect, the step of providing the digital representation D₃₋ₓ of each further coating layer CL-x being present in the substrate in addition to the coating layer CL may comprise:
providing a measure indicating the permittivity of the further coating layer CL-x being present or
providing data derived from the chemical composition of the coating material used to prepare the further layer CL-x and/or providing data on least one physical property of the coating material used to prepare the further layer CL-x and/or providing data on least one physical property of the further coating layer CL-x.

Further coating layer(s) being present in addition to coating layer CL and their respective digital representation(s) are generally designated by CL-x and D₃₋ₓ, wherein the x is replaced by other appropriate letters in the naming of the specific individual coating layer and digital representation. If, for example, exactly one further coating layer, such as a clearcoat layer, is present, it is denoted as CL-1. The corresponding digital representation is denoted as D₃₋₁. If two further coating layers, such as a primer layer and a clearcoat layer, are present, they are denoted as CL-1 and CL-2 respectively. The corresponding digital representations are denoted as D₃₋₁ and D₃₋₂ respectively. Providing the aforementioned data in step (i) may include manual input of said data or importing said data from a computer readable medium, such as a database or a computer file. Data derived from the chemical composition of the coating material may include the type and amount of component being present in the coating material. Data on the property of the coating material used to prepare the further coating layer(s) may include, for example, the solids content of said coating material. Data on the further coating layer(s) CL-x may include, for example, appearance data, such as flop index data; color values; data describing the orientation of effect pigments, preferably aluminum pigments, within the coating layer(s) CL-x; data acquired during the application of the coating material used to prepare the coating layer(s) CL-x; the conductivity of the coating layer(s) CL-x and combinations thereof. In case the further coating layer(s) CL-x does/do not contain any pigments being known to have an influence on the transmission and/or reflection of electromagnetic radiation, it might be sufficient to provide a standard measure indicating the permittivity as digital representation D₃₋ₓ. The term “standard measure indicating the permittivity” refers to a measure indicating the permittivity, which is representative for the respective further coating layer CL-x, such as, e.g. a primer layer or a clearcoat layer. In case the further coating layer(s) CL-x does/do contain effect pigments, such as aluminum pigments, the digital representation(s) D₃₋ₓ preferably comprises at least data derived from the chemical composition of the coating materials used to prepare the further coating layer(s) CL-x. In this regard, it may be further preferred if data on at least one physical property of the coating material(s) and the coating layer(s) CL-x is provided in combination with data derived from the chemical composition of the coating material(s), because this may increase the accuracy of the determination of the measure indicating the permittivity of the coating layer(s) CL-x and therefore also the accuracy of the proposed method.

### Step (ii):

In step (ii), a data-driven model is provided which is parametrized on digital representations Dₕ of historical coating layers and historical measures indicating the permittivity of said coating layers. The data-driven model provides a relationship between the measure indicating the permittivity and the properties of the coating layer and is derived from the digital representations Dₕ of historical coating layers and historical measures indicating the permittivity of said coating layers. The properties of the coating layer may be chemical properties and/or physical properties. Chemical properties may include the type and amounts of components being present in the coating material used to prepare the coating layer. Physical properties may include data on at least one physical property of the coating material as well as data on at least one physical property of the coating layer. In particular, the digital representation Dₕ comprises the formulation of the coating material used to prepare the historical coating layer, physical property data of the coating material, such as the solid content, and physical property data of the historical coating layer, such as flop index data, color values, data describing the orientation of effect pigments, preferably aluminum pigments, within the historical coating layer, data acquired during the application of the coating material used to prepare the historical coating layer and combinations thereof.

In an aspect, the data driven model may be a rigorous model, an empirical model, or a combination thereof, preferably a rigorous model. The rigorous model may be developed by determining relationships between the measure indicating the permittivity and the data on the properties of historical coating materials and coating layers prepared from these materials. An empirical model may be developed by using an artificial intelligence model as described later on to determine these relationships. The model may be trained by providing digital representations Dₕ of historical coating layers and historical measures indicating the permittivity of said coating layers to said model.

Step (iii):

In step (iii) of the proposed method, the measure indicating the permittivity of the coating layer CL is determined based on the data-driven model and the digital representation D₁ of the coating layer CL. In an aspect, the data-driven model provides a relationship between at least one descriptor D and the measure indicating the permittivity. In a preferred example, the relationship is a linear relationship. In another example, the relationship is a non-linear relationship, such as a polynomial relationship. Said descriptor D describes the influence of the amount and type of pigment, preferably effect pigment, in relation to the solids content of coating material on the measure indicating the permittivity. The descriptor D may further describe the influence of further components except pigments, being present in the coating material and/or the influence of properties of the coating layer CL or further coating layer(s) CL-x on the measure indicating the permittivity. Properties of the coating layers CL and CL-x may include chemical and/or physical properties, for example appearance, such as flop index, color values, the orientation of the effect pigments within the respective coating layer, data acquired during the application of the coating material used to prepare the respective coating layer, the conductivity and combinations thereof.

Considering the influence of the further components except pigments and/or the influence of the properties of the coating layer CL or further coating layer(s) CL-x on the measure indicating the permittivity may improve the accuracy of the relationship and thus may provide a better prediction of the at least one transmission and/or reflection property of the coated substrate.

The descriptor D is calculated from a pigment content descriptor D_{PIG} and optionally from a component descriptor D_{R} and/or a property descriptor D_{PROP}. In one example, the descriptor D is calculated from D_{PIG} and D_{R} and/or D_{PROP} by multiplication of D_{PIG} with D_{R} and/or D_{PROP}. In another example, the descriptor D is calculated from D_{PIG} and D_{R} and/or D_{PROP} by addition of D_{PIG} with D_{R} and/or D_{PROP}.

The pigment content descriptor D_{PIG} may be obtained by formula (I)

$${D}_{PIG} ( A , {W}_{PIG} , S ) = \frac{\sum {A}^{{W}_{PIG}}}{S} \qquad ( I )$$

in which
A represents the % by weight—based on the total weight of the coating material—of pigment, preferably aluminum pigment, being present in the coating material,
S represents the solids content of the coating material in % by weight, and
W_{PIG} represents the pigment weighing factor.

In case the coating material used to prepare the coating layer CL contains aluminum pigment in combination with further pigments known to have no significant influence on the transmission and/or reflection properties of the coated substrate, the pigment descriptor D_{PIG} may be obtained by formula (Ia)

$${D}_{PIG} ( A , {W}_{PIG} , S ) = \frac{{A}^{{W}_{PIG}}}{S} \qquad ( Ia )$$

in which
A represents the % by weight—based on the total weight of the coating material—of aluminum pigment being present in the coating material and W_{PIG} and S have the same meaning as in formula (I).

The pigment weighing factor W_{PIG} describes the influence of each pigment on the measure indicating the permittivity and may be derived, for example, from the BET surface of the pigments and/or from the particle size of the pigments. This factor can be derived by determining the measure indicating the permittivity of the coating layer and correlating this measure with the properties of the pigments being present in the coating material used to prepare this coating layer.

The component descriptor D_{R} may be obtained by formula (II)

$${D}_{R} ( {A}_{R} , {W}_{R} , n ) = \frac{\sum {A}_{R}^{{W}_{R}}}{n} \qquad ( II )$$

in which
A_{R} represents the % by weight—based on the total weight of the coating material—of each component except pigment being present in the coating material,
n represents the number of components being present in the coating material, and
W_{R} represents the component weighing factor.

The component weighing factor W_{R} describes the influence of each component (except pigments) present in the coating material on the measure indicating the permittivity. In case of polymers being present in the coating material, said factor may be derived from the measure indicating the permittivity of said polymers. The measure indicating the permittivity of said polymers can, for example, be derived from the structure of the polymer. For this purpose, the crystallinity and/or the presence of functional groups can be considered.

The property descriptor D_{PROP} may be obtained by formula (III)

$${D}_{P R O P} ( P , {W}_{P} , n ) = \frac{\sum {P}^{{W}_{PROP}}}{n} \qquad ( III )$$

in which
P represents the property of the coating layer CL,
n represents the number of properties used to calculate D_{PROP}, and
W_{PROP} represents the property weighing factor.

The property weighing factor W_{PROP} describes the influence of each property of the coating layer on the measure indicating the permittivity. This factor can be derived by determining the measure indicating the permittivity of the coating layer and correlating this measure with the respective property. Properties, which may be considered include the appearance, such as the flop index or sparkle intensity, color data, such as color space data, the orientation of effect pigments in the coating layer CL or the type of application of the coating material used to prepare the coating layer CL. One example of color space data are defined by L*a*b*, where L* represents luminous intensity, a* represents a red/green appearance, b* represents a yellow/blue appearance. Another example of color space data is defined by L*, C*, h, where L* represents lightness, C* represents chroma, and h represents hue.

### Optional Step (iv):

In optional step (iv), the measure indicating the permittivity of at least one further coating layer CL-x may be determined based on the data-driven model and the digital representation D₃₋ₓ of the coating layer. In case more than one further coating layer CL-x is present, optional step (iv) may be performed for each further coating layer CL-x being present. In another example, optional step (iv) may be performed for a part of all further coating layers CL-x being present. Optional step (iv) is preferably performed for further coating layer(s) CL-x, which contain pigments and/or further components known in the state of the art to have a significant influence on the measure indicating the permittivity. This increases the accuracy of the proposed method because the use of a standard measure indicating the permittivity might not be sufficiently consider the presence of such pigments and/or further components. Step (iv) is preferably performed as previously described in connection with step (iii) by using the respective digital representation D₃₋ₓ instead of the digital representation D₁.

In an aspect, the measure indicating the permittivity may be selected from the relative permittivity εᵣ.

### Optional Step (v):

In optional step (v) of the proposed method, at least one transmission and/or reflection property of the coating layer CL or the coated substrate is determined based on at least the measure indicating the permittivity of the coating layer CL provided as previously described and the digital representation D₂ of the coated substrate. Step (v) can be performed according to different alternatives which are listed in a non-limiting manner in the following.

According to a first alternative, at least one transmission and/or reflection property of the coating layer CL is determined based on the measure indicating the permittivity provided in step (iii) and the digital representation D₂ of the coated substrate. This allows to screen different coating formulations with respect to the fulfillment of certain requirements, such as required transmission and/or reflection properties of the resulting coating layers CL. For this purpose, the same reference substrate, i.e. the same permittivity of a substrate, is used during determination of the transmission and/or reflection properties of the respective coating layers CL.

According to a second alternative, at least one transmission and/or reflection property of a coated substrate comprising at least two coating layers, i.e. the coating layer CL and at least one further coating layer CL-x, is determined based on the measure indicating the permittivity provided in step (iii), the digital representation D₂ of the coated substrate and the measure indicating the permittivity of each further coating layer CL-x. The measure indicating the permittivity of each further coating layer CL-x being present on the substrate can be obtained in numerous ways.

In one example, the measure indicating the permittivity is retrieved for each further coating layer CL-x from the respective digital representation D₃₋ₓ provided in step (i).

This may be preferred if the measure indicating the permittivity of all further coating layer(s) CL-x has been previously determined by experiments or if a standard measure indicating the permittivity can be used and said measures are contained in each respective provided digital representation D₃₋ₓ.

In another example, the measure indicating the permittivity as determined in step (iv) is used for all further coating layers CL-x in step (v). This option may be used in case none of the provided digital representations D₃₋ₓ contains the measure indicating the permittivity required in step (v).

In yet another example, the measure indicating the permittivity as determined in step (iv) may be used for part of the further coating layers CL-x while said measure is retrieved from provided respective digital representation D₃₋ₓ for the remaining further coating layers CL-x. This may be beneficial if the measure indicating the permittivity has been determined experimentally for only a part of the further coating layer(s) CL-x or a standard measure can be used for said part while said measure is unknown and thus needs to be determined in step (iv) for the remaining further coating layer(s) CL-X.

In an aspect of optional step (v), the at least one transmission and/or reflection property of the coated substrate may be selected from (i) the transmission spectra, (ii) the damping in transmission, preferably the one-way and/or two-way damping in transmission, (iii) the reflection spectra, (iv) the damping in reflection, and (v) combinations thereof. In one example, the transmission spectra and the reflection spectra may each be calculated using the transfer matrix method. This method is well known in the state of the art and is based on the fact that, according to Maxwell's equations, there are simple continuity conditions for the electric field across boundaries from one medium to the next. If the field is known at the beginning of a layer, the field at the end of the layer can be derived from a simple matrix operation. A stack of layers can then be represented as a system matrix, which is the product of the individual layer matrices. The final step of the method involves converting the system matrix back into reflection and transmission coefficients.

The transmission and reflection spectra may each be calculated in a frequency range that is commonly used in combination with coated substrates. Since the frequency range can be freely chosen, the proposed method is universally applicable to all coated substrates which are used in combination with devices emitting electromagnetic radiation and detecting the reflected electromagnetic radiation. In case the coated substrates are used in the automotive sector in combination with radar sensing devices, a frequency range of 15 to 300 GHz may be used. In one example, the transmission and reflection spectra may therefore each be calculated in a frequency range of 15 to 300 GHz, preferably in a frequency range of 15 to 150 GHz, very preferably in a frequency range of 15 to 40 GHz and/or in a frequency range of 60 to 90 GHz and/or in a frequency range of 125 to 155 GHz. The damping in transmission, preferably the one-way and/or two-way damping in transmission, and the damping in reflection may each be obtained from the transmission and reflection spectra at a frequency of 24 GHz and/or at a frequency of 76.5 GHz and/or at a frequency of 137 GHz.

### Step (vi):

In step (vi), the measure indicating the permittivity of the coating layer CL determined as previously described and/or the at least one transmission and/or reflection property determined as previously described is provided via a communication interface. Providing said measure or the at least one property may include displaying said measure or the at least one property via a display to a user. The display may comprise a GUI in order to increase user comfort. The determined measure or property may be transferred to a computer readable medium, such as a database, for storage. The determined measure or property may be provided to a computer processor to be used in further steps run on said processor. This may be particularly preferred if the proposed method includes optional step (v) or further steps as described in the following.

Further steps:

In an aspect, the proposed method may further include the steps of
(vii) optionally determining if the at least one transmission and/or reflection property provided in step (vi) is within at least one predefined transmission and/or reflection tolerance;
(viii) optionally providing via the communication interface the result of the determination performed in step (vii);
(ix) optionally providing recommendations via the communication interface if the at least one transmission and/or reflection property provided in step (vi) is outside of the predefined transmission and/or reflection tolerance;
(x) optimizing the at least one transmission and/or reflection property provided in step (vi) by modifying the digital representation D₁ and/or the digital representation D₂ and/or the digital representation D₃₋ₓ provided in step (i) until the predefined transmission and/or reflection tolerance is reached;
(xi) providing via the communication interface the optimized digital representation D₁ and/or the digital representation D₂ and/or the digital representation D₃₋ₓ and the optimized least one transmission and/or reflection property of the coated substrate.

Optional step (vii) may include comparing the at least one transmission and/or reflection property provided in step (vi) with at least one predefined transmission and/or reflection tolerance. The tolerance may be a numerical value or may be a numerical range and may be defined by the user manually before performing step (vii) or may be stored on a computer readable medium, such as a database. In one example, the predefined transmission and/or reflection tolerance may describe the value of a damping in transmission and/or reflection, that should not be exceeded in order to provide acceptable performance of a radar sensing device mounted behind the coated substrate. The comparison may be done manually or automatically. Manual comparison may be performed by a person and may include comparing the property/properties provided in step (vi) to a tolerance known to the user. Automatic comparison may be performed by the at least one processor and may either be initiated by a user after the at least one transmission and/or reflection property is provided via the communication interface to the user or may be started automatically after the at least one transmission and/or reflection property of the coated substrate has been determined, for example by automatically providing said property/properties via the communication interface to at least one processor and performing the comparison.

In optional step (viii), the result of the determination performed in optional step (vii) may be provided via the communication interface. This may be preferred if the at least one transmission and/or reflection property provided in step (vi) is compared automatically to the predefined transmission and/or reflection tolerance. The result of the determination may be displayed via the communication interface to a user. In another example, the result of the determination may be provided via the communication interface to at least one processor or a computer readable medium, such as a database. This may be preferred if recommendations are to be provided in case the at least one property provided in step (vi) is outside of the predefined transmission and/or reflection tolerance.

In optional step (ix), recommendations may be provided via the communication interface if the at least one transmission and/or reflection property provided in step (vi) is outside of the predefined transmission and/or reflection tolerance. The recommendations may be stored on a computer readable medium, such as a database. In an example, at least one processor may access a database containing the recommendations and may retrieved respective recommendations based on the result of the determination in step (vii) provided via the communication interface to the processor. Said retrieved recommendations may then be displayed to the user via the communication interface comprising a display, in particular a display including a GUI. An example recommendation may be “Radar requirements not fulfilled. Please modify the thickness of substrate and/or at least one layer thickness of a coating layer being present on the substrate”. Another example recommendation may be “Radar requirements not fulfilled. Please modify the coating composition.”

In step (x), the at least one transmission and/or reflection property provided in step (vi) is optimized by modifying the digital representation D₁ and/or the digital representation D₂ and/or the digital representation D₃₋ₓ provided in step (i) until the predefined transmission and/or reflection tolerance is reached.

In one example, modifying the digital representation D₁ and/or the digital representation D₂ and/or the digital representation D₃₋ₓ in step (x) may include manipulating at least one adjustment tool of a plurality of adjustment tools displayed on the communication interface comprising a display having a graphical user interface, each of the adjustment tools corresponding to a particular digital representation D₁, D₂ and optionally D₃₋ₓ provided in step (i). The digital representation D₁ and/or the digital representation D₂ and/or the digital representation D₃₋ₓ provided in step (i) may be displayed via the adjustment tools by setting the regulator to a position corresponding to said digital representations. Modifying may then be performed by a user by moving at least one regulator, for example via a computer mouse or a finger (in case the display includes a touchscreen), of at least one adjustment tool. In addition to displaying at least one regulator, numerical value(s) may be displayed for each digital representation provided in step (i). This value(s) may be automatically updated in response to moving the regulator to provide an interactive guidance of the optimizing process to the user.

Modifying the digital representation D₁ and/or D₃₋ₓ in step (x) may include providing a digital representation D₁ₘ and/or D₂ₘ and/or D₃₋ₓₘ being different from the digital representation D₁ and/or D₂ and/or D₃₋ₓ provided in step (i) and optionally automatically moving adjustment tools displayed on the communication interface comprising a display having a graphical user interface in response to the provided digital representation D₁ₘ and/or D₂ₘ and/or D₃₋ₓₘ. The modified digital representations D₁ₘ and/or D₂ₘ and/or D₃₋ₓₘ may be provided by importing said digital representations from a computer readable medium, such as a database. After providing said modified digital representations, the user may further manipulate the updated adjustment tools as previously described.

In another example, modifying the digital representation D₁ and/or the digital representation D₂ and/or the digital representation D₃₋ₓ in step (x) may include conducting a search in at least one database containing digital representations D₁ₕ and D₃ₕ₋ₓ of historical coating layers and/or digital representations D₂ₕ of historical coated substrates in connection with at least one transmission and/or reflection property. The results of the search may be displayed on the communication interface comprising a display to the user and the user may select an appropriate result. The displayed results may be sorted according to their relevance. The relevance may be calculated using predefined criteria and may provide a guidance to the user. Alternatively, the closest match may be automatically selected and may be used to modify the respective digital representation. The closest match may be determined according to predefined criteria.

In yet another example, modifying the digital representation D₁ and/or D₃₋ₓ in step (x) includes obtaining a digital representation D₁ₘ and/or D₃₋ₓₘ having an acceptable color deviation from the digital representation D₁ and/or D₃₋ₓₘ provided in step (i). This may be particularly preferred, if the coating layer CL and/or the further coating layer(s) CL-x are used to provide the substrate with a specific visual impression, for example with a specific color and/or appearance, e.g. if the coating layer CL and optionally at least one further coating layer CL-x is used as basecoat layers.

In one example, obtaining the digital representation D₁ₘ and/or D₃₋ₓₘ having an acceptable color deviation may include determining a proposed coating formulation and associated proposed color values, calculating the differences between the color values of the digital representation D₁ and/or D₃₋ₓ provided in step (i) and the proposed color values to define differential color values, inputting the color values of the digital representation D₁ and/or D₃₋ₓ provided in step (i) and the differential color values into an artificial intelligence model and determining if the proposed color solution is acceptable by utilizing the artificial intelligence model. The color values may include color space values, reflectance values or other suitable color attributes. One example of color space values is defined by L*a*b*, where L* represents luminous intensity, a* represents a red/green appearance, b* represents a yellow/blue appearance. Another example of color space values is defined by L*, C*, h, where L* represents lightness, C* represents chroma, and h represents hue. The color values for the digital representation D₁ and/or D₃₋ₓ and the color values of the proposed coating formulation may be obtained using a multi-angle or spherical geometry color measuring device, a spectrophotometer, digital camera, or other suitable device.

The step of determining the proposed coating formulation and associated proposed color values may be further defined as searching a database for the proposed color solution based on the color values of the digital representation D₁ and/or D₃₋ₓ provided in step (i). For this purpose, the color values of the digital representation D₁ and/or D₃₋ₓ may be provided via the communication interface comprising a display having a GUI, for example by inputting this color values by the user or by importing these color values from a computer readable medium, such as a file or a database.

The differences between the color values of the digital representation D₁ and/or D₃₋ₓ provided in step (i) and the proposed color values are calculated by utilizing the computer to define differential color values. The differential color values are typically expressed as ΔL*, ΔC*, Δh* or ΔL*, Δa*, Δb*. The calculation to determine the differential color values may be accomplished using any suitable mathematical calculation as is known in the art.

The differential color values are then input into the artificial intelligence model. The inputting of these values focuses and assists the artificial intelligence model to determine the most accurate acceptability rating for the proposed coating formulation.

This example may further include the step of training the artificial intelligence model for determining acceptability. The method of training the artificial intelligence model may include the initial step of inputting color values of the digital representation D₁ and/or D₃₋ₓ provided in step (i) and differential color values to the input layer of the neural network. A proposed coating formulation associated with the inputted color values of the digital representation D₁ and/or D₃₋ₓ provided in step (i) and the inputted differential color values is also inputted into the artificial intelligence model. The artificial intelligence model now has all the required information and a numerical output, indicative of the acceptability of the proposed coating formulation, is produced. A weighted factor to the color values is used to determine the acceptability of the numerical output. The step of training the artificial intelligence model may include the step of comparing the output to a known acceptability of the proposed color solution. For this purpose, the numerical output is fed into a comparator. A known acceptability of the proposed coating formulation is first converted into a known numerical output and then the known acceptability is also inputted to the comparator. The known acceptability is a previously determined and known acceptability rating for the proposed coating formulation that was inputted into the artificial intelligence model. The comparator compares the output of the artificial intelligence model with the previously known acceptability of the proposed coating formulation and produces an error value. If the artificial intelligence model is fully trained and operating properly, the error value will be negligible, and no further action would be taken. However, if the artificial intelligence model is in the process of being trained, then the error value will be relatively large. The error value is compared to an error limit to determine error variation. If the error value exceeds the error limit, then error feedback is provided to the artificial intelligence model corresponding to the error variation. The weighted factor is then adjusted according to the error feedback. Typically, this training procedure will be initiated for hundreds or even thousands of different inputs in order to adequately train the artificial intelligence model.

The artificial intelligence model may be embodied in a neural network. More specifically, the artificial intelligence model may be a back propagation neural network where feedback is provided to the neural network from the output. Neural network techniques are a member of a group of methods which fall under the umbrella of artificial intelligence. Artificial intelligence is commonly associated with logic rule-based expert systems where the rule hierarchies used are reasoned from human knowledge. In contrast, neural networks are self-trained based on experience acquired through data compilation and computation. The neural network may include an input layer and an output layer. The input layer has input nodes and the output layer has output nodes. Each output node corresponds with an input node. Between the input and output layers, there may be one or more hidden layers, each having one or more hidden nodes corresponding to an input node and output node pair. Each input variable is associated with an input node and each output variable is associated with an output node. More specifically, a node receives the input, processes this input, and provides an output. The processing step includes summing the inputs, adding a bias value, and submitting this total input to an activation function which limits the magnitude of the output. The connections between the various nodes are weighted. An output sent from one node to another is multiplied by the weighting factor associated between those two particular nodes. The weighting factor represents the knowledge of the system and is preferably adjusted during training by providing feedback from the output to the input layer. A suitable neural network is, for example, disclosed in U.S. Pat. No. 7,536,231 B2.

The output of the artificial intelligence model, in particular the neural network, which is indicative of the acceptability of the proposed coating formulation may be transformed into any desired format. For example, the output could be transformed into a numerical variable indicative of the acceptability of the output. The numerical variable could be a single continuous variable that may assume any value between two endpoints. An example being the set of real numbers between 0 and 1. As a further example, the numerical variable could consider the uncertainty inherent in the data, for example in the color measurement data and the output of the neural network. An example being the range from 0 to 1, with a 1 indicating no uncertainty in the result. The output could also be transformed into a descriptive output indicative of the acceptability of the output. In particular, the descriptive output could include an acceptable/moderately acceptable/not acceptable format, an acceptance factor format, or any other suitable format. The output may be provided via the communication interface comprising a display having a GUI to the user.

This example may further include the step of providing via the communication interface the digital representation D₁ₘ and/or D₃₋ₓₘ having an acceptable color deviation from the digital representation D₁ and/or D₃₋ₓ provided in step (i). In particular, the modified coating formulation may be provided on a display having a GUI.

If the proposed coating formulation is determined to be outside a range of acceptability, one or more additional steps could occur. For example, a diagnostic or error type message could be determined and sent to the user to assist the user in modifying the inputs provided in step (i) or (x). Step (x) as discussed above is then repeated.

In another example, obtaining a digital representation D₁ₘ and/or D₃₋ₓₘ having an acceptable color deviation from the digital representation D₁ and/or D₃₋ₓₘ provided in step (i) may include modifying the formulation of the coating material(s) used to prepare the coating layer CL and/or the coating layer(s) CL-x, preparing the modified coating material(s), applying and curing said modified coating material(s), obtaining the color values of said cured coting layer(s) and determining whether the color values are within a predefined tolerance from the color values of the digital representation D₁ and/or D₃₋ₓ. Modifying the formulation of the coating material(s) may be performed by manipulating at least one adjustment tool or by performing a search in a database as previously described. If the obtained color values are outside of the predefined threshold, such as ΔE>1, the coating formulation is adapted, for example by changing the concentration of pigments such that the predefined threshold is met. Afterwards, the respective digital representation is modified to correspond to the adapted coating formulation and the transmission and/or reflection property is calculated as previously described in order to ensure that the predefined transmission and/or reflection tolerance is met for the adapted coating material. In case the predefined tolerance is not met, this process is repeated.

In step (xi), the at least one optimized transmission and/or reflection property of the coated substrate is provided. This may include automatic updating of the at least one transmission and/or reflection property provided in step (vi) in response to optimizing the at least one transmission and/or reflection property in step (x). Providing the optimized transmission and/or reflection property/properties may include displaying said property/properties on a display comprising a GUI. This may allow an interactive guidance of the user, in particular if step (x) is performed by manipulating at least one adjustment tool or by importing at least one modified digital representation D₁ and/or D₂ and/or D₃₋ₓ from a computer readable medium.

The proposed method further including at least steps (x) and (xi) allows to modify the digital representations D₁, D₂ and optionally D₃₋ₓ to optimize the transmission and/or reflection properties, in particular the damping in radar transmission and/or reflection, until a predefined transmission and/or reflection tolerance is reached. The modified digital representation D₁ and optionally D₃₋ₓ, in particular the chemical composition, may be checked with regard to an acceptable color tolerance with respect to the digital representation D₁ and optionally D₃₋ₓ provided in step (i), thus allowing to select coating materials providing the same visual appearance as the digital representation D₁ and optionally D₃₋ₓ provided in step (i) but having transmission and/or reflection properties within the predefined tolerances. This is especially useful if the selected coating material does not fulfill the predefined transmission and/or reflection tolerance and needs to be adapted without a visual impact on the resulting color. This is furthermore especially useful for refinish purposes where coating materials need to be selected which can be used to repair trim parts comprising a multilayer coating having defective sites without resulting in a non-acceptable visible appearance as well as a non-acceptable dampening of the radar intensity of the multilayer coating formed after the repair process.

### Embodiments of the Inventive Apparatus

In an aspect, the computing apparatus may further comprise at least one measuring device connected via the communication interface to the processing module. Suitable measuring devices may include color measuring devices such as multi-angle or spherical geometry color measuring devices, spectrophotometers, digital cameras and/or devices to determine data on the property/properties of coating materials. This may allow to provide the respective data directly to the processing module and thus reduces the amount of input required by the user.

In an aspect, the computing apparatus may further comprise at least one database DB1 connected via the communication interface to the processing module, said database DB1 containing a digital representation D₁ of the coating layer CL and/or a digital representation D₂ of the coated substrate and/or a digital representation D₃₋ₓ of each further coating layer being present on the substrate in addition to the coating layer CL and/or vehicle identification data. This may allow to easily select the required data based on the vehicle identification and reduces manual input prone to errors. IN case the vehicle identification number is scanned from a tag, the system may further comprise at least one reader, such as bar code or QR code reader.

In an aspect, the communication interface may comprise a display, in particular a display having a graphical user interface, in particular a graphical user interface comprising at least one adjustment tool. This may allow an interactive guidance of the user during data input and also allows to display the predicted measure indicating the permittivity of the coating layer CL or the predicted transmission and/or reflection spectra of the coated substrate

In an aspect, the computing apparatus may further comprise at least one database DB2 connected via the communication interface to the processing module, said database DB2 containing a digital representation D₁ of the coating layer CL and/or a digital representation D₂ of the coated substrate and/or a digital representation D₃₋ₓ of each further coating layer being present on the substrate in addition to the coating layer CL in connection with at least one transmission and/or reflection property. This may be preferred if step (x) of the proposed method is performed by conducting a search in at least one database containing the previously mentioned data.

In an aspect, the computing apparatus may further comprise at least one database DB3 connected via the communication interface to the processing module, said database DB3 containing coating formulations and associated color values. This may be preferred, if step (x) of the proposed method is performed by obtaining a digital representation D₁ₘ and/or D₃₋ₓₘ having an acceptable color deviation from the digital representation D₁ and/or D₃₋ₓ provided in step (i).

In an aspect, the computing apparatus may further comprise at least one database DB4 connected via the communication interface to the processing module, said database DB4 containing the data-driven model. This may be preferred if the data-driven model is a rigorous model.

In an alternative aspect, the processing module may comprise at least one artificial intelligence module. This may be preferred, if step (x) of the proposed method is performed by obtaining a digital representation D₁ₘ and/or D₃₋ₓₘ having an acceptable color deviation from the digital representation D₁ and/or D₃₋ₓ provided in step (i)

### Embodiments of the Inventive Client Device

In an aspect of the inventive client device, the server device corresponds to the inventive apparatus described previously.

Further embodiments or aspects are set forth in the following numbered clauses:
1. A computer-implemented method for predicting the properties of a coating layer CL or the transmission and/or reflection properties of a substrate being coated with a coating layer CL and optionally at least one further coating layer CL-x, said method comprising the steps of:
(i) providing to a computer processor via a communication interface a digital representation D₁ of the coating layer CL, optionally a digital representation D₂ of the coated substrate and optionally a digital representation D₃₋ₓ of each further coating layer CL-x being present on the substrate in addition to the coating layer CL;
(ii) providing to the computer processor via the communication interface a data driven model parametrized on
   digital representations Dₕ of historical coating layers, and
   historical measures indicating the permittivity of said coating layers;
(iii) determining with the computer processor a measure indicating the permittivity of the coating layer CL based on
   the data driven model provided in step (ii), and
   the digital representation D₁ of the coating layer CL;
(iv) optionally determining with the computer processor a measure indicating the permittivity of at least one further coating CL-x layer being present on the substrate in addition to the coating layer CL based on
   the data driven model provided in step (ii), and
   the digital representation D₃₋ₓ of the further coating layer CL-x;
(v) optionally determining with the computer processor at least one transmission and/or reflection property of the coated substrate based on
   the measure indicating the permittivity of the coating layer CL provided in step (iii),
   optionally the digital representation D₃₋ₓ of each further coating layer CL-x being present on the substrate in addition to the coating layer CL or the measure indicating the permittivity of the at least one further coating layer CL-x provided in step (iv) optionally in combination with the digital representation D₃₋ₓ of further coating layers CL-x for which the measure indicating the permittivity was not provided in step (iv), and
   the digital representation D₂ of the coated substrate;
(vi)) providing via the communication interface the determined measure indicating the permittivity of the coating layer CL and/or the determined at least one transmission and/or reflection property of the coated substrate.
2. The method according to clause 1, wherein the substrate is transparent to electromagnetic radiation having a frequency of 22 to 300 GHz, preferably a frequency of 22 to 144 GHz.
3. The method according to clause 1 or 2, wherein the substrate is comprising or consisting of polycarbonate, blends of polycarbonate and polybutylene terephthalate, elastomer-modified polypropylene, blends of polypropylene and ethylene-propylene-diene rubber, acrylonitrile butadiene styrene copolymer, blends of acrylonitrile butadiene styrene copolymer with polycarbonate, acryl ester styrene acrylonitrile copolymer, polyamide and blends thereof, polyurethanes, blends of polycarbonate and polyethylene terephthalate, polybutylene terephthalate and mixtures thereof.
4. The method according to any of the preceding clauses, wherein the coating layer CL is selected from pigmented coating layers, preferably from basecoat layers.
5. The method according to any of the preceding clauses, wherein the substrate is coated with a multilayer coating comprising the following layers, in particular in the stated order: optionally at least one primer layer PL, the coating layer CL, in particular being a basecoat layer, optionally at least one further basecoat layer being different from the coating layer CL and at least one clearcoat layer CL.
6. The method according to any of the preceding clauses, wherein the communication interface comprises a display, preferably a display having a graphical user interface.
7. The method according to any of the preceding clauses, wherein providing the digital representation D₁ of the coating layer CL and/or the digital representation D₂ of the coated substrate and/or the digital representation D₃₋ₓ of each further coating layer CL-x in step (i) includes providing vehicle identification data, obtaining the digital representation D₁ and/or D₂ and/or D₃₋ₓ based on the provided vehicle identification data, and providing said obtained digital representation D₁ and/or D₂ and/or D₃₋ₓ.
8. The method according to clause 7, wherein the step of obtaining the digital representation D₁ and/or D₂ and/or D₃₋ₓ is further defined as searching a database for said digital representations based on the inputted vehicle identification data.
9. The method according to any of the preceding clauses, wherein providing the digital representation D₁ of the coating layer CL comprises providing data derived from the chemical composition of the coating material used to prepare the coating layer CL,
   optionally providing data on least one physical property of the coating material used to prepare the coating layer CL, and
   optionally providing data on least one physical property of the coating layer CL.
10. The method according to clause 9, wherein data derived from the chemical composition of the coating material includes the type and amount of each pigment, in particular effect pigment, being present in the coating material.
11. The method according to clause 9 or 10, wherein providing data derived from the chemical composition of the coating material comprises importing the formulation of said coating material from a computer readable medium, in particular at least one database.
12. The method according to any of clauses 9 to 11, wherein data on at least one physical property of the coating material is selected from the solids content of said coating material.
13. The method according to any of clauses 9 to 11, wherein data on at least one physical property of the coating layer CL is selected from (i) appearance data, such as flop index data; (ii) color values; (iii) data describing the orientation of effect pigments, preferably aluminum pigments, within the coating layer CL; (iv) data acquired during the application of the coating material; and (v) combinations thereof, preferably flop index data.
14. The method according to any of the preceding clauses, wherein providing the digital representation D₂ of the coated substrate comprises providing the thickness of the substrate, a measure indicating the permittivity of the substrate, the layer thickness of the coating layer CL and optionally of each further coating layer CL-x being present on the substrate in addition to the coating layer CL.
15. The method according to any of the preceding clauses, wherein providing the digital representation D₃₋ₓ of each further coating layer CL-x being present in the substrate in addition to the coating layer CL comprises
   providing a measure indicating the permittivity of the further coating layer CL-x being present or
   providing data derived from the chemical composition of the coating material used to prepare the further layer CL-x and/or providing data on least one physical property of the coating material used to prepare the further layer CL-x and/or providing data on least one physical property of the further coating layer CL-x.
16. The method according to any of the preceding clauses, wherein the data driven model is a rigorous model, an empirical model, or a combination thereof, preferably a rigorous model.
17. The method according to any of the preceding clauses, wherein the data-driven model provides a relationship, in particular a linear relationship, between at least one descriptor D and the measure indicating the permittivity, said descriptor D describing the influence of the amount and type of pigment, preferably effect pigment, in relation to the solids content of coating material on the measure indicating the permittivity and optionally the influence of further components of the coating material and/or the influence of properties of the coating layer CL or further coating layer(s) CL-x on the measure indicating the permittivity.
18. The method according to clause 17, wherein the descriptor D is calculated from a pigment content descriptor D_{PIG} and optionally from a component descriptor D_{R} and/or a property descriptor D_{PROP}.
19. The method according to clause 18, wherein the pigment content descriptor D_{PIG} is obtained by formula (I)

$${D}_{PIG} ( A , {W}_{PIG} , S ) = \frac{\sum {A}^{{W}_{PIG}}}{S} \qquad ( I )$$

in which
A represents the % by weight—based on the total weight of the coating material—of pigment, preferably aluminum pigment, being present in the coating material,
S represents the solids content of the coating material in % by weight, and
W_{PIG} represents the pigment weighing factor.
20. The method according to clause 18 or 19, wherein the component descriptor D_{R} is obtained by formula (II)

$${D}_{R} ( {A}_{R} , {W}_{R} , n ) = \frac{\sum {A}_{R}^{{W}_{R}}}{n} \qquad ( II )$$

in which
A_{R} represents the % by weight—based on the total weight of the coating material—of each component except pigment being present in the coating material,
n represents the number of components being present in the coating material, and
W_{R} represents the component weighing factor.
21. The method according to any of clauses 18 to 20, wherein the property descriptor D_{PROP} is obtained by formula (III)

$${D}_{P R O P} ( P , {W}_{P} , n ) = \frac{\sum {P}^{{W}_{P R O P}}}{n} \qquad ( III )$$

in which
P represents the property of the coating layer CL,
n represents the number of properties used to calculate D_{PROP}, and
W_{PROP} represents the property weighing factor.
22. The method according to any of the preceding clauses, wherein the measure indicating the permittivity is selected from the relative permittivity εᵣ.
23. The method according to any of the preceding clauses, wherein the at least one transmission and/or reflection property of the coated substrate is selected from (i) the transmission spectra, (ii) the damping in transmission, preferably the one-way and/or two-way damping in transmission, (iii) the reflection spectra, (iv) the damping in reflection, and (v) combinations thereof.
24. The method according to clause 23, wherein the transmission spectra and reflection spectra are each calculated using the transfer matrix method
25. The method according to clause 23 or 24, wherein the transmission spectra and the reflection spectra are each calculated in a frequency range of 15 to 300 GHz, preferably in a frequency range of 15 to 150 GHz, very preferably in a frequency range of 15 to 40 GHz and/or in a frequency range of 60 to 90 GHz and/or in a frequency range of 125 to 155 GHz.
26. The method according to any of clauses 23 to 25, wherein the damping in transmission, preferably the one-way and/or two-way damping in transmission, and the damping in reflection are each calculated at a frequency of 24 GHz and/or at a frequency of 76.5 GHz and/or at a frequency of 137 GHz.
27. The method according to any of the preceding clauses, wherein the step of providing the determined measure indicating the permittivity of the coating layer CL and/or the determined at least one transmission and/or reflection property includes displaying said determined measure or at least one property on a display and/or storing said determined measure or at least one property on a computer-readable medium and/or providing said determined measure or at least one property to a computer processor.
28. The method according to any of the preceding clauses further including the steps of
   (vii) optionally determining if the at least one transmission and/or reflection property provided in step (vi) is within at least one predefined transmission and/or reflection tolerance;
   (viii) optionally providing via the communication interface the result of the determination performed in step (vii);
   (ix) optionally providing recommendations via the communication interface if the at least one transmission and/or reflection property provided in step (vi) is outside of the predefined transmission and/or reflection tolerance;
   (x) optimizing the at least one transmission and/or reflection property provided in step (vi) by modifying the digital representation D₁ and/or the digital representation D₂ and/or the digital representation D₃₋ₓ provided in step (i) until the predefined transmission and/or reflection tolerance is reached;
   (xi) providing via the communication interface the optimized digital representation D₁ and/or the digital representation D₂ and/or the digital representation D₃₋ₓ and the optimized least one transmission and/or reflection property of the coated substrate.
29. The method according to clause 28, wherein modifying the digital representation D₁ and/or the digital representation D₂ and/or the digital representation D₃₋ₓ in step (x) includes manipulating at least one adjustment tool of a plurality of adjustment tools displayed on the communication interface comprising a display having a graphical user interface, each of the adjustment tools corresponding to a particular digital representation D₁, D₂ and optionally D₃₋ₓ provided in step (i).
30. The method according to clause 28 or 29, wherein modifying the digital representation D₁ and/or D₂ and/or D₃₋ₓ in step (x) includes providing a digital representation D₁ₘ and/or D₂ₘ and/or D₃₋ₓₘ being different from the digital representation D₁ and/or D₂ and/or D₃₋ₓ provided in step (i) and optionally automatically moving adjustment tools displayed on the communication interface comprising a display having a graphical user interface in response to the provided digital representation D₁ₘ and/or D₂ₘ and/or D₃₋ₓₘ.
31. The method according to clause 28, wherein modifying the digital representation D₁ and/or the digital representation D₂ and/or the digital representation D₃₋ₓ in step (x) includes conducting a search in at least one database containing digital representations D₁ₕ and D₃ₕ₋ₓ of historical coating layers and/or digital representations D₂ₕ of historical coated substrates in connection with the at least one transmission and/or reflection property.
32. The method according to clause 28, wherein modifying the digital representation D₁ and/or D₃₋ₓ in step (x) includes obtaining a digital representation D₁ₘ and/or D₃₋ₓₘ having an acceptable color deviation from the digital representation D₁ and/or D₃₋ₓ provided in step (i).
33. The method according to clause 32, wherein obtaining a digital representation D₁ₘ and/or D₃₋ₓₘ having an acceptable color deviation includes determining a proposed coating formulation and associated proposed color values, calculating the differences between the color values of the digital representation D₁ and/or D₃₋ₓ provided in step (i) and the proposed color values to define differential color values, inputting the color values of the digital representation D₁ and/or D₃₋ₓ provided in step (i) and the differential color values into an artificial intelligence model and determining if the proposed coating formulation is acceptable by utilizing the artificial intelligence model.
34. The method according to clause 33, wherein the step of determining the proposed coating formulation and associated proposed color values is further defined as searching a database for the proposed color solution based on the color values of the digital representation D₁ and/or D₃₋ₓ provided in step (i).
35. The method according to clause 33 or 34, further including the step of training the artificial intelligence model for determining acceptability.
36. The method according to clause 35, wherein the step of training the artificial intelligence model includes the step of comparing the output to a known acceptability of the proposed color solution.
37. The method according to clause 35 or 36, wherein the artificial intelligence model is a neural network and further including the step of providing feedback to the neural network from the output.
38. The method according to clause 37, wherein the neural network includes an input layer and an output layer and further including the step of providing feedback from the output to the input layer.
39. The method according to any of clauses 33 to 38, further including the step of providing via the communication interface the digital representation D₁ₘ and/or D₃₋ₓₘ having an acceptable color deviation from the digital representation D₁ and/or D₃₋ₓ provided in step (i).
40. The method according to any of clauses 28 to 39, wherein providing at least one optimized transmission and/or reflection property of the coated substrate in step (xi) includes automatically updating the at least one transmission and/or reflection property provided in step (vi) in response to optimizing the at least one transmission and/or reflection property in step (x).
41. A computing apparatus comprising:
   a communication interface;
   a processing module comprising at least one computer processor; and
   a memory storing instructions that, when executed by the processing module, configure the system to perform the steps of the computer implemented method according to any of clauses 1 to 40.
42. The computing apparatus according to clause 41, further comprising at least one measuring device connected via the communication interface to the processing module.
43. The computing apparatus according to clause 41 or 42, further comprising at least one database DB1 connected via the communication interface to the processing module, said database DB1 containing a digital representation D₁ of the coating layer CL and/or a digital representation D₂ of the coated substrate and/or a digital representation D₃₋ₓ of each further coating layer being present on the substrate in addition to the coating layer CL and/or vehicle identification data.
44. The computing apparatus according to any of clauses 41 to 43, wherein the communication interface comprises a display, in particular a display having a graphical user interface, in particular a graphical user interface comprising at least one adjustment tool.
45. The computing apparatus according to any of clauses 41 to 44, further comprising at least one database DB2 connected via the communication interface to the processing module, said database DB2 containing a digital representation D₁ of the coating layer CL and/or a digital representation D₂ of the coated substrate and/or a digital representation D₃₋ₓ of each further coating layer being present on the substrate in addition to the coating layer CL in connection with at least one transmission and/or reflection property.
46. The computing apparatus according to any of clauses 41 to 45, further comprising at least one database DB3 connected via the communication interface to the processing module, said database DB3 containing coating formulations and associated color values.
47. The computing apparatus according to any of clauses 41 to 46, further comprising at least one database DB4 connected via the communication interface to the processing module, said database DB3 containing a data-driven model.
48. The computing apparatus according to any of clauses 41 to 47, wherein the processing module comprises at least one artificial intelligence module.
49. A non-transitory computer-readable storage medium, the computer-readable storage medium including instructions that when executed by a computer, cause the computer to perform the steps according to any of clauses 1 to 40.
50. A system comprising:
   at least one coating layer CL; and
   at least one measure indicating the permittivity of said at least one coating layer CL, wherein said measure indicating the permittivity is determined according to the method of any of clauses 1 to 40.
51. Use of the method of any of clauses 1 to 40 for screening coating layers CL or coated substrates comprising at least one coating layer CL and optionally at least one further coating layer CL-x according to at least one criterion.
52. A substrate being coated with a coating layer CL and at least one further coating layer CL-x, wherein the transmission and/or reflection property of the substrate was derived according to the method of any of clauses 1 to 40.
53. A client device for generating a request to initiate the prediction of at least one property of a coating layer CL or at least one transmission and/or reflection property of a substrate being coated with a coating layer CL and optionally at least one further coating layer CL-x at a server device, wherein the client device is configured to provide a digital representation D₁ of the coating layer CL, optionally a digital representation D₂ of the coated substrate, optionally a digital representation D₃₋ₓ of each further coating layer CL-x being present on the substrate in addition to the coating layer CL and optionally a tolerance to a server device.
54. The client device of embodiment 53, wherein the server device is an apparatus according to any one of embodiments 41 to 48.

### BRIEF DESCRIPTION OF THE DRAWINGS

These and other features of the present invention are more fully set forth in the following description of exemplary embodiments of the invention. To easily identify the discussion of any particular element or act, the most significant digit or digits in a reference number refer to the figure number in which that element is first introduced. The description is presented with reference to the accompanying drawings in which:

FIG. 1 is a block diagram of a method for predicting the transmission and reflection properties of a substrate being coated with a coating layer CL and optionally at least one further coating layer CL-x

FIG. 2 is a block diagram of a preferred embodiment of the inventive method

FIG. 3 illustrates a computing apparatus in accordance with the invention

FIG. 4 illustrates client server setup for the proposed methods

FIG. 5 is a planar view of an input screen that is partially populated with data

FIG. 6 is a planar view of an output screen illustrating an adjustment tool and is populated with data and displaying the transmission and reflectance spectra as well as damping in transmission and reflection

### DETAILED DESCRIPTION

The detailed description set forth below is intended as a description of various aspects of the subject-matter and is not intended to represent the only configurations in which the subject-matter may be practiced. The appended drawings are incorporated herein and constitute a part of the detailed description. The detailed description includes specific details for the purpose of providing a thorough understanding of the subject-matter. However, it will be apparent to those skilled in the art that the subject-matter may be practiced without these specific details.

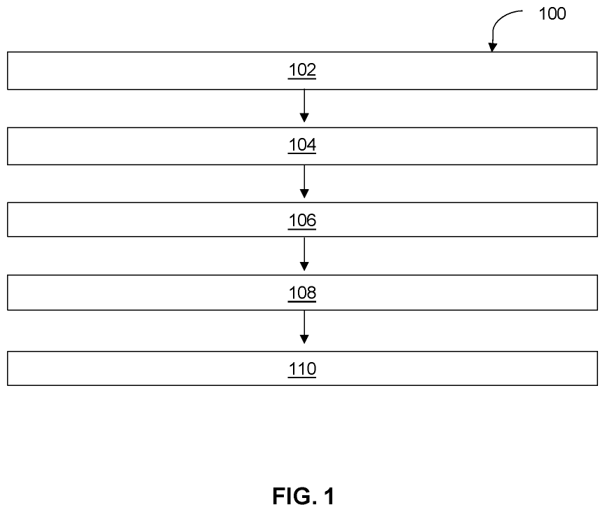

FIG. 1 depicts a non-limiting embodiment of the computer implemented method of predicting the transmission and reflection properties of a substrate being coated with a coating layer CL and optionally at least one further coating layer CL-x. In this example, the coating layer CL is a basecoat layer comprising aluminum effect pigments and the substrate further comprises a clearcoat layer.

In block 102, routine 100 provides to at least one computer processor via a communication interface the digital representation D₁ of the basecoat layer CL and the digital representation D₂ of the coated substrate. In this example, the digital representation D₃₋₁ of the clearcoat layer being present in addition to the basecoat layer is provided to the at least one processor via the communication interface, this step being generally optional. In this example, providing the digital representation D₁ of the basecoat layer CL comprises providing the following data: the amount of aluminum pigment (in percent by weight, based on the total weight of the coating material) in the coating material used to prepare the basecoat layer CL, the solids content of the coating material used to prepare the basecoat layer CL, and the flop index of the basecoat layer CL. In this example, providing the digital representation D₂ of the coated substrate comprises providing the following data: permittivity εᵣ of a standard substrate, thickness of the substrate, layer thickness of the basecoat layer CL, layer thickness of the clearcoat layer CL-1. In another example, the permittivity εᵣ of the substrate used/to be used in combination with the basecoat layer CL is provided. In this example, providing the digital representation D₃₋₁ of the clearcoat layer CL-1 comprises providing the permittivity εᵣ of a standard clearcoat layer. In accordance with the invention, a permittivity εᵣ of a specific substrate or specific clearcoat layer may be provided. According to an alternative, the digital representation D₃₋₁ of the clearcoat layer may comprise similar data than the digital representation D₁. This may be preferred if the clearcoat layer comprise pigments known to have an influence on the permittivity εᵣ of the clearcoat layer.

In block 104, routine 100 provides to the at least one computer processor via the communication interface a data driven model parametrized on digital representations Dₕ of historical basecoat layers and historical measures of the permittivity εᵣ of said basecoat layers. In this example, the digital representations Dₕ of historical basecoat layers comprise the formulation, e.g. the ingredients and amounts of ingredients, solids content and solids density of the coating materials used to prepare the historical basecoat layers, as well as the flop index, the film thickness and the conductivity of the historical basecoat layers. In this example, the data driven model is a rigorous model obtained from comparing the digital representations Dₕ of historical basecoat layers with their respective permittivity εᵣ and provides a linear relationship between at least one descriptor D and the permittivity εᵣ of the basecoat layer. The descriptor D is calculated in this example from a pigment content descriptor D_{PIG} according to formula (I)

$${D}_{PIG} ( A , {W}_{PIG} , S ) = \frac{{A}^{{W}_{PIG}}}{S} \qquad ( I )$$

in which
A represents the % by weight—based on the total weight of the coating material—of aluminum pigment being present in the coating material,
S represents the solids content of the coating material in % by weight, and
W_{PIG} represents the pigment weighing factor.

In block 106, routine 100 determines with the at least one computer processor the permittivity εᵣ of the basecoat layer CL based on the data driven model provided in block 104 and the digital representation D₁ of the basecoat layer CL provided in block 102. For this purpose, the descriptor D described is determined from the digital representation D₁ of the basecoat layer CL as previously described and used in the data driven model to obtain the permittivity εᵣ of the basecoat layer CL.

In block 108, routine 100 determines with at least one computer processor the transmission and reflection spectra as well as the damping in transmission and reflection of the coated substrate based on the permittivity εᵣ of the basecoat layer CL determined in block 106, the permittivity εᵣ of the clearcoat layer CL-1 provided via the digital representation D₃₋₁ as well as the permittivity εᵣ of a standard substrate, the thickness of the substrate, the layer thickness of the basecoat layer CL and the layer thickness of the clearcoat layer CL-1 provided via the digital representation D₂ of the coated substrate.

In block 110, routine 100 provides the transmission and reflection spectra as well as the damping in transmission and reflection determined in block 108 via the communication interface.

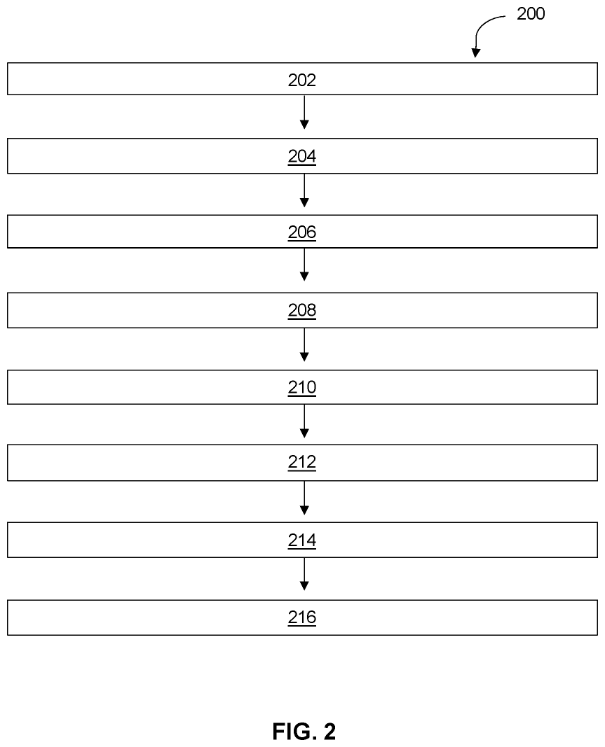

FIG. 2 depicts a further non-limiting embodiment of the computer implemented method of predicting the transmission and reflection properties of a substrate being coated with a coating layer CL and optionally at least one further coating layer CL-x. In this embodiment, at least one digital representation provided in block 202 is modified until a predefined transmission and reflection tolerance is met. In this example, the coating layer CL is a basecoat layer comprising aluminum effect pigments. The steps performed in blocks 202 to 210 correspond to the steps performed in blocks 102 to 110 described in connection with FIG. 1.

In block 202, routine 200 provides to at least one computer processor via a communication interface the digital representation D₁ of the basecoat layer CL, the digital representation D₂ of the coated substrate and the digital representation D₃₋₁ of the clearcoat layer being present in addition to the basecoat layer as described in connection with block 102 of FIG. 1.

In block 204, routine 200 provides to the at least one computer processor via the communication interface a data driven model parametrized on digital representations Dₕ of historical basecoat layers and historical measures of the permittivity εᵣ of said basecoat layers as described in connection with block 104 of FIG. 1.

In block 206, routine 200 determines with the at least one computer processor the permittivity εᵣ of the basecoat layer CL based on the data driven model provided in block 204 and the digital representation D₁ of the basecoat layer CL provided in block 202 as described in connection with block 106 of FIG. 1.

In block 208, routine 200 determines with at least one computer processor the transmission and reflection spectra as well as the damping in transmission and reflection of the coated substrate based on the permittivity εᵣ of the basecoat layer CL determined in block 206, the permittivity εᵣ of the clearcoat layer CL-1 provided via the digital representation D₃₋₁ as well as the permittivity εᵣ of a standard substrate, the thickness of the substrate, the layer thickness of the basecoat layer CL and the layer thickness of the clearcoat layer CL-1 provided via the digital representation D₂ of the coated substrate.

In block 210, routine 200 provides the transmission and reflection spectra as well as the damping in transmission and reflection determined in block 208 via the communication interface.

In block 212, routine 200 determines if the damping in transmission and reflection provided in block 210 is within a predefined damping in transmission and reflection tolerance. In this example, this is determined by a user by comparing the provided damping in transmission and reflection with a predefined tolerance. The predefined tolerance might be, for example, a maximum damping in transmission of −2 dB. In another example, said comparison can be performed automatically by routine 200 on the at least one computer processor. In this case, the predefined tolerance may be stored on a computer readable medium, such as a database, and may be accessed by the computer processor to perform the comparison.

In block 214, routine 200 optimizes the damping in transmission and reflection provided in block 210 by modifying the digital representation D₁ and/or the digital representation D₂ and/or the digital representation D₃₋₁ provided in block 202 until the predefined damping in transmission and reflection tolerance is met. In this example, the digital representation D₂ of the coated substrate is modified by manipulating a virtual adjustment tool comprising different regulators for the thickness of the substrate and the layer thickness of the basecoat layer CL and the clearcoat layer CL-1. For this purpose, the regulator displaying the current thickness of the substrate is moved by a user by clicking on a regulator and moving said regulator until the damping in transmission is below the tolerance or threshold given in block 212. In another example, the digital representation D₁ and/or D₃₋ₓ may be modified. This may be performed by manipulating virtual adjustment tools, by searching a database or by obtaining a modified digital representation D₁ₘ and/or D₃₋ₓₘ having an acceptable color deviation from the digital representation D₁ and/or D₃₋ₓ provided in block 202.

In block 216, routine 200 provides via the communication interface the optimized digital representation D₁ₘ and/or the digital representation D₂ₘ and/or the digital representation D₃₋ₓₘ and the optimized at least one transmission and/or reflection property. The communication interface may comprise a display including a GUI. In this example, the optimized thickness of the substrate and the optimized damping in transmission and reflection is provided via the communication interface comprising a display to the user. In another example, the optimized digital representation D₁ₘ and/or D₃₋ₓₘ and the optimized at least one transmission and/or reflection property is provided via the communication interface.

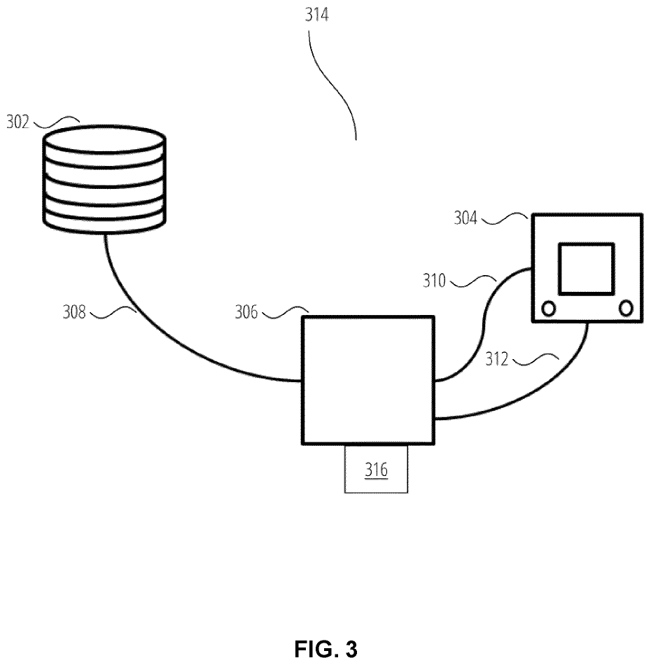

FIG. 3 shows an example of a computing apparatus 300 comprising: a computer processor 306; a communication interface 308, 310, 312, and a memory 316 storing instructions that, when executed by the processor, configure the apparatus to perform the steps of
providing to a computer processor via a communication interface a digital representation D₁ of the basecoat layer CL, a digital representation D₂ of the coated substrate and a digital representation D₃₋₁ of a further clearcoat layer CL-1 being present on the substrate in addition to the basecoat layer CL;
providing to the computer processor via the communication interface a data driven model parametrized on digital representations Dₕ of historical coating layers and historical measures indicating the permittivity εᵣ of said historical coating layers;
determining with the computer processor the permittivity εᵣ of the basecoat layer CL based on the provided data driven model provided in step and the digital representation D₁ of the basecoat layer CL;
determining with the computer processor transmission and reflection properties of the coated substrate based on the permittivity εᵣ of the basecoat layer CL, a standard permittivity εᵣ for the clearcoat layer CL-1 and the digital representation D₂ of the coated substrate; and
providing via the communication interface the determined transmission and reflection properties of the coated substrate.

In this example the computer apparatus further comprises in input/output device 304.

In this example the data driven model is stored in a database 302. The database 302 is connected to the computer processor via the communication interface 308. In this example input/output device 304 is used to provide the digital representations D₁ and D₃₋₁ of the coating layer CL and CL-1 to the computer processor 306 via communication interface 310. In this example the digital representation D₁ is provided in the form of a chemical composition of the coating material used to prepare the basecoat layer CL, the solid content of the coating material and the flop index of the basecoat layer CL. In this example, the digital representation D₂ is provided in the form of a εᵣ of a standard substrate, the thickness of the substrate, the layer thickness of the basecoat layer CL and the layer thickness of the clearcoat layer CL-1. The data driven model is provided to the computer processor 306 via the communication interface 308. With the computer processor 306 transmission and reflection properties are determined. In this example the transmission and reflection properties are provided to the input/output device 304 via communication interface 312. In another example the transmission and reflection properties may be provided to the data base 302 via communication interface 308.

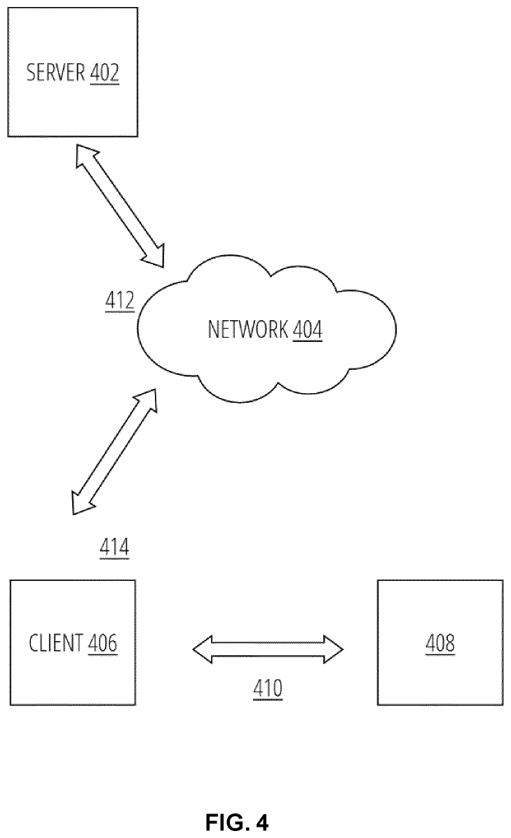

Turning to FIG. 4, there is shown an Internet-based system for predicting at least one transmission and/or reflection property of a substrate being coated with a coating layer CL and optionally at least one further coating layer CL-x. The system 400 comprises a server 402 which can be accessed via a network 404, such as the Internet, by one or more clients 406.1 to 406.n. Preferably, the server may be an HTTP server and is accessed via conventional Internet web-based technology. The clients 406 are computer terminals accessible by a user and may be customized devices, such as data entry kiosks, or general-purpose devices, such as a personal computer. A printer 408 can be connected to a client terminal 406. The internet-based system is in particular useful, if a service is provided to customers or in a larger company setup. A client may be used to provide the digital representations D₁, D₂ and optionally D₃₋ₓ to the computer processor of the server.

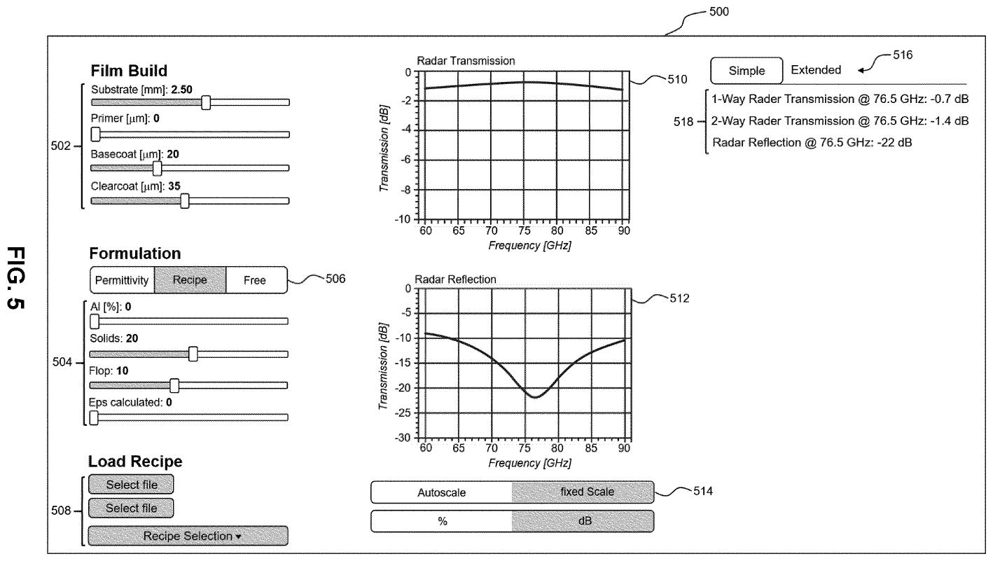

FIG. 5 illustrates a graphical user interface 500 that is displayed to the user upon start of the method, for example upon start of routine 100 or 200 of FIGS. 1 and 2. This graphical user interface 500 could be displayed on any device, such as portable and stationary devices, comprising a display. In this example, the GUI is displayed on a computer display. The graphical user interface 500 includes a number of adjustment tools 502, 504 and buttons 506, 508 for inputting a variety of data which may be used to predict the transmission and/or reflection properties of the coated substrate and a number of areas 510, 512, 518 for displaying the predicted transmission and/or reflection properties of the coated substrate.

In this example, various adjustment tools 502, 504 each having various regulators which can be moved by a computer mouse or a finger (in case the display comprises a touchscreen) to provide data with respect to the coated substrate and the coating layer CL are displayed on the GUI 500. In order to increase user guidance during use of the adjustment tools 502, 504, the value corresponding to the actual position of the regulator is given above the respective regulator and is automatically updated during movement of the regulator by the user. The values displayed in adjustment tools 502, 504 are placeholders and need to be adjusted by the user, if necessary. In another example, buttons for importing data from files or data entry fields may be used in instead or in combination with adjustment tool 502. In this example, part of the digital representation D₂ is provided by using adjustment tool 502. In this example, a fixed permittivity εᵣ of a standard substrate is used and cannot be provided via adjustment tool 502. In another example, the permittivity εᵣ can be entered by the user using the adjustment tool 502 by modifying the respective regulator. In this example, adjustment tool 504 can be used to provide data on the formulation of the coating material and on the properties of the resulting coating layer CL. In this example, the coating layer CL is a basecoat layer. In another example, the GUI 500 may comprise more than one adjustment tool 504, such as 504.1 to 504.n, to allow the user to provide data on the formulation of the coating material and on the properties of the resulting coating layer(s) CL-x. The data provided in adjustment tools 504.1 to 504.n may be used as digital representation(s) D₃₋ₓ. This is preferred if, for example, two different basecoat layers are used and the permittivity εᵣ of each basecoat layer needs to be determined from the provided data.

In this example, area 506 comprises three different buttons which allow the user to switch between different input modes of data in adjustment tool 504 by hitting the respective button. In the “recipe mode” (i.e. when the button “Recipe” is activated), the user can perform the following actions:
automatically set the values for the aluminum content, the solid content and the flop index by importing a formulation of the coating material used to prepare the coating layer CL and further property data, i.e. the solid content of the coating material and the flop index of the coating layer CL, by clicking on the button(s) “Select file” in area 508. After recipe import, the regulators are automatically moved to the values of the imported data. The regulator “Al [%]” is fixed after recipe import, i.e. the amount cannot be adjusted using this regulator, while regulators for the solid content and the flop index can still be moved as desired after recipe import or
automatically set the values for the aluminum content or the solid content or the flop index by data import as previously described while adjusting the remaining values by moving the respective regulators.

The permittivity εᵣ of the coating layer CL is determined in the “recipe mode” based on the entered data and the regulator “eps calculated” is automatically moved to the display the determined value.

In the “permittivity mode” (i.e. when button “Permittivity” in area 506 is activated), the user can only input the desired permittivity εᵣ of the coating layer CL by moving the regulator “eps calculated” in area 504 while the other regulators in this area are blocked, and the transmission and reflection properties are predicted merely on the basis of the inputted permittivity.

In the “free mode” (i.e. when button “Free” in area 506 is activated), the user can move all regulators displayed in area 504 to enter the respective values. In case no recipe is imported, only the values for the aluminum content, the solid content and the flop index being shown in adjustment tool 504 are used to predict the transmission and reflection property even if a permittivity is inputted. The user can also import a recipe as previously described but in the “free mode”, the aluminum content can still be adjusted by the user after import of the recipe.

In this example, areas 508 and 510 comprise the transmission spectra and the reflection spectra of the coated substrate in a frequency range of 60 to 90 GHz. The data displayed in areas 510 and 512 is a placeholder which is automatically adjusted once the user starts entering data in area 502 and/or 504. In area 514 the user can choose whether the graphs displayed in area 510 and 512 have a fixed frequency and transmission range (button “fixed Scale”) or whether the displayed graphs have a transmission range which is scaled automatically to increase user comfort (button “Autoscale”). In this example, the graphs displayed in area 510 and 512 have a fixed frequency and transmission range because the button “Fixed Scale” is activated.

In this example, area 518 comprises the damping in transmission and reflection at a frequency of 76.5 GHz and the radar reflection which is obtained from the transmission and reflection spectra in areas 510 and 512. The data displayed in this area is placeholder data which is updated automatically if the user starts entering data in area 502 and/or 504. In area 516, the user can choose whether the damping in transmission and reflection and the radar reflection are displayed (tab “Simple”) or whether further information, such as values are used for calculation of descriptor D, type of effect pigment being present in the highest amounts, etc., is displayed (tab “Extended”). In this example, tab “Simple” is activated.

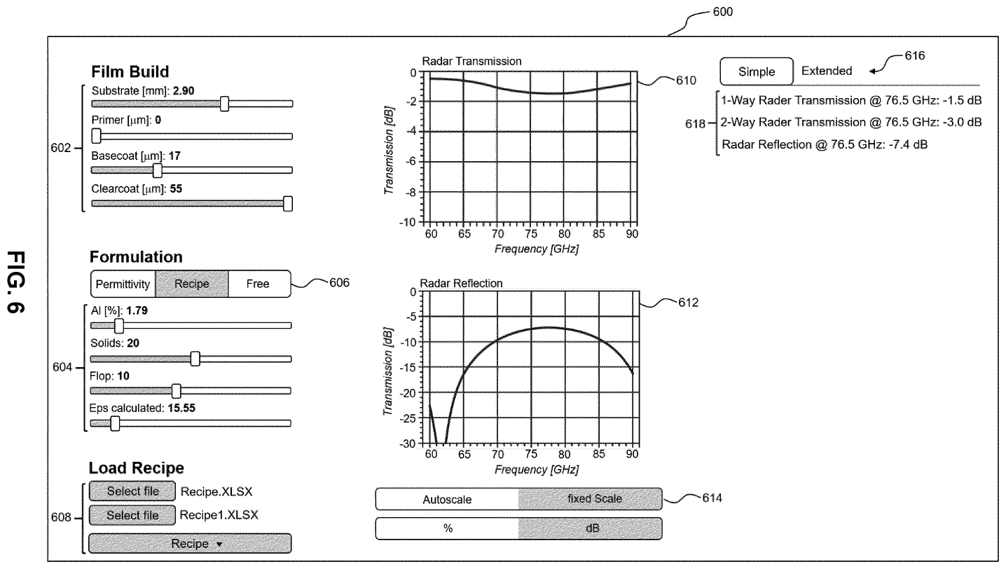

FIG. 6 illustrates a graphical user interface 600 that is displayed to the user after a recipe has been uploaded. This graphical user interface 600 could be displayed on any suitable portable or stationary device having a display. The graphical user interface 600 includes a number of adjustment tools 602, 604, displaying the provided data and a number of areas 610, 612, 618 displaying the predicted transmission and/or reflection properties of the coated substrate based on the provided data.

In this example, the thickness of the substrate, the basecoat layer and the clearcoat layer has been adjusted by moving the regulators of adjustment tool 602 to the respective positions.

In this example, a recipe has been uploaded by clicking on the button “Select file” in area 608 and the regulator “Al [%]” of adjustment tool 604 has been moved to the amount listed in the imported recipe. Based on this amount, the permittivity εᵣ of the basecoat layer CL is determined and the regulator “eps calculated” of adjustment tool 604 is automatically moved to the display the determined value of 15.55.

In this example, the placeholder transmission and reflection spectra of FIG. 5 are updated based on the data inputted via adjustment tools 602 and 604 and are displayed in areas 610 and 612, respectively. Moreover, the placeholders “damping in transmission and reflection at 76.5 GHz” as well as “radar reflection at 76.5 GHz” are also updated and the updated values are displayed in area 618.

1. A computer-implemented method for predicting the properties of a coating layer CL or the transmission and/or reflection properties of a substrate coated with a coating layer CL and optionally at least one further coating layer CL-x, said method comprising the steps of:
(i) providing to a computer processor via a communication interface a digital representation D₁ of the coating layer CL, optionally a digital representation D₂ of the coated substrate and optionally a digital representation D₃₋ₓ of each further coating layer CL-x is present on the substrate in addition to the coating layer CL;
(ii) providing to the computer processor via the communication interface a data driven model parametrized on
digital representations Dₕ of historical coating layers, and
historical measures indicating the permittivity of said coating layers;
(iii) determining with the computer processor a measure indicating the permittivity of the coating layer CL based on
the data driven model provided in step (ii), and
the digital representation D₁ of the coating layer CL;
(iv) optionally determining with the computer processor a measure indicating the permittivity of at least one further coating CL-x layer present on the substrate in addition to the coating layer CL based on
the data driven model provided in step (ii), and
the digital representation D₃₋ₓ of the further coating layer CL-x;
(v) optionally determining with the computer processor at least one transmission and/or reflection property of the coated substrate based on
the measure indicating the permittivity of the coating layer CL provided in step (iii),
optionally the digital representation D₃₋ₓ of each further coating layer CL-x present on the substrate in addition to the coating layer CL or the measure indicating the permittivity of the at least one further coating layer CL-x provided in step (iv) optionally in combination with the digital representation D₃₋ₓ of further coating layers CL-x for which the measure indicating the permittivity was not provided in step (iv), and
the digital representation D₂ of the coated substrate; and
(vi) providing via the communication interface the determined measure indicating the permittivity of the coating layer CL and/or the determined at least one transmission and/or reflection property of the coated substrate.

2. The method according to claim 1, wherein the coating layer CL is selected from pigmented coating layers.

3. The method according to claim 1, wherein providing the digital representation D₁ of the coating layer CL and/or the digital representation D₂ of the coated substrate and/or the digital representation D₃₋ₓ of each further coating layer CL-x in step (i) comprises providing vehicle identification data, obtaining the digital representation D₁ and/or D₂ and/or D₃₋ₓ based on the provided vehicle identification data, and providing said obtained digital representation D₁ and/or D₂ and/or D₃₋ₓ.

4. The method according to claim 1, wherein providing the digital representation D₁ of the coating layer CL comprises the steps of:
providing data derived from the chemical composition of the coating material used to prepare the coating layer CL,
optionally providing data on least one physical property of the coating material used to prepare the coating layer CL, and
optionally providing data on least one physical property of the coating layer CL.

5. The method according to claim 1, wherein providing the digital representation D₂ of the coated substrate comprises providing the thickness of the substrate, a measure indicating the permittivity of the substrate, the layer thickness of the coating layer CL and optionally of each further coating layer CL-x being is present on the substrate in addition to the coating layer CL.

6. The method according to claim 1, wherein providing the digital representation D₃₋ₓ of each further coating layer CL-x is present in the substrate in addition to the coating layer CL comprises the steps of:
providing a measure indicating the permittivity of the further coating layer CL-x is present, or
providing data derived from the chemical composition of the coating material used to prepare the further layer CL-x and/or providing data on least one physical property of the coating material used to prepare the further layer CL-x and/or providing data on least one physical property of the further coating layer CL-x.

7. The method according to claim 1, wherein the data driven model is selected from the group consisting of a rigorous model, an empirical model, and a combination thereof.

8. The method according to claim 1, wherein the data-driven model provides a relationship between at least one descriptor D and the measure indicating the permittivity, said descriptor D describing the influence of the amount and type of pigment in relation to the solids content of coating material on the measure indicating the permittivity and optionally the influence of further components of the coating material and/or the influence of properties of the coating layer CL or further coating layer(s) CL-x on the measure indicating the permittivity.

9. The method according to claim 7, wherein the descriptor D is calculated from a pigment content descriptor D_{PIG} and optionally from a component descriptor D_{R} and/or a property descriptor D_{PROP}.

10. The method according to claim 1 further comprising the steps of:
(vii) optionally determining if the at least one transmission and/or reflection property provided in step (vi) is within at least one predefined transmission and/or reflection tolerance;
(viii) optionally providing via the communication interface the result of the determination performed in step (vii);
(ix) optionally providing recommendations via the communication interface if the at least one transmission and/or reflection property provided in step (vi) is outside of the predefined transmission and/or reflection tolerance;
(x) optimizing the at least one transmission and/or reflection property provided in step (vi) by modifying the digital representation D₁ and/or the digital representation D₂ and/or the digital representation D₃₋ₓ provided in step (i) until the predefined transmission and/or reflection tolerance is reached; and
(xi) providing via the communication interface the optimized digital representation D₁ and/or the digital representation D₂ and/or the digital representation D₃₋ₓ and the optimized least one transmission and/or reflection property of the coated substrate.

11. A computing apparatus comprising:
a communication interface;
a processing module comprising at least one computer processor; and
a memory storing instructions that, when executed by the processing module, configure the system to perform the steps of the computer implemented method according to claim 1.

12. A non-transitory computer-readable storage medium, wherein the computer-readable storage medium including instructions that when executed by a computer, cause the computer to perform the steps according to claim 1.

13. A method of using a computer-implemented method of claim 1, wherein the method comprising using the method for screening coating layers CL or coated substrates comprising at least one coating layer CL and optionally at least one further coating layer CL-x according to at least one criterion.

14. A substrate coated with a coating layer CL and at least one further coating layer CL-x, wherein the transmission and/or reflection property of the substrate was derived according to the method of claim 1.

15. A client device for generating a request to initiate the prediction of at least one property of a coating layer CL or at least one transmission and/or reflection property of a substrate coated with a coating layer CL and optionally at least one further coating layer CL-x at a server device, wherein the client device is configured to provide a digital representation D₁ of the coating layer CL, optionally a digital representation D₂ of the coated substrate, optionally a digital representation D₃₋ₓ of each further coating layer CL-x being present on the substrate in addition to the coating layer CL and optionally a tolerance to a server device.

16. The method according to claim 1, wherein the coating layer CL is selected from basecoat layers.

17. The method according to claim 1, wherein the data driven model is a rigorous model.

18. The method according to claim 1, wherein the data-driven model provides a linear relationship between at least one descriptor D and the measure indicating the permittivity, said descriptor D describing the influence of the amount and type of effect pigment, in relation to the solids content of coating material on the measure indicating the permittivity and optionally the influence of further components of the coating material and/or the influence of properties of the coating layer CL or further coating layer(s) CL-x on the measure indicating the permittivity.
